(19) United States
(12) Patent Application Publication
(10) Pub. No.: US 2012/0076722 A1
(43) Pub. Date: Mar. 29, 2012

(54) METHODS FOR TREATING CANCERS AND DISEASES ASSOCIATED WITH 4-1BB (CD137) EXPRESSION

(75) Inventors: Scott Strome, Reistertown, MD (US); Xiaoyu Zhang, Catonsville, MD (US)

(73) Assignee: UNIVERSITY OF MARYLAND, BALTIMORE, Baltimore, MD (US)

(21) Appl. No.: 13/320,267

(22) PCT Filed: May 11, 2010
(86) PCT No.: PCT/US10/34295

§ 371 Date: Dec. 14, 2011

Related U.S. Application Data
(60) Provisional application No. 61/178,347, filed on May 14, 2009.

(51) Int. Cl. A61K 39/395; A61K 51/00; C12Q 1/68; G01N 21/75; G01N 33/566; A61P 37/04; A61K 38/17

(52) U.S. Cl. 424/1.11; 424/173.1; 424/278.1; 435/7.1; 436/501; 435/6.12

(57) ABSTRACT

The present invention relates to the role of 4-1BB (CD137) ligand and anti-4-1BB (CD137) antibody in the treatment of cancers and diseases associated with 4-1BB (CD137) expression. More particularly, the present invention relates to the use of (i) 4-1BB (CD137) ligand for inducing proliferation and activation and promoting survival of B lymphocytes and (2) anti-4-1BB (anti-CD137) antibody for inhibiting proliferation and activation and inducing death of B lymphocytes.

### BACKGROUND OF THE INVENTION

4-1BB (CD137/ILA) is a member of the TNF receptor superfamily and is predominantly found on activated T cells and NK cells (1-3). 4-1BB (CD137) ligand (4-1BBL or CD137L) is present on antigen presenting cells including dendritic cells, macrophages, monocytes and B cells (4-5). Stimulation of 4-1BB, through either its natural ligand or agonistic antibody (anti-4-1BB or anti-4-1BB antibody), induces potent anti-tumor immunity (6-8), yet also effectively ameliorates disease severity in several mouse models of autoimmunity, including systemic lupus erythematosus (9), chronic graft versus host disease (10), collagen induced arthritis (11, 12), inflammatory bowel disease (13), and experimental autoimmune encephalitis (14). Thus, immunotherapeutics targeting 4-1BB represent promising new approaches to a wide array of distinct immune disorders.

The explanation for the apparent disparity between the ability to promote tumor rejection and treat autoimmune disease appears to be predicated on 4-1BB-mediated manipulation of T cell function. Specifically, in conceptually overlapping experimental models of autoimmunity, 4-1BB ligation induces T cell deletion or hyporesponsiveness (10, 13), stimulation of CD4⁺CD25⁺ antigen-specific regulatory T cell subsets (15) or proliferation of antigen-specific CD8⁺ CD11c⁺ T cells, which suppress CD4⁺ T cell responses (11). This 4-1BB-mediated immune modulation of T cells is postulated to be mechanistically responsible for observed changes in B cell function, including diminished isotype-specific antibody responses and changes in B cell survival. For example, in both murine and primate models, administration of agonistic antibodies against 4-1BB reduces T cell-dependent antibody production; 4-1BB deficient mice demonstrate reduced IgG2a and IgG3 responses to KLH. (16-18). Additionally, the importance of 4-1BB-4-1BBL co-stimulation in B cell survival is evidenced in 4-1BBL transgenic mice, where B cells are noted to decline in absolute number with advancing age (19). Importantly, murine B cells do not express 4-1BB, while human B cells are reported to up-regulate 4-1BB in response to anti-IgM stimulation (20, 21). Therefore, it is uncertain whether murine-based animal models will accurately predict clinical response to 4-1BB manipulation. Considering the importance of B cells in anti-tumor immune regulation and autoimmunity (22, 23), it is striking that the function of 4-1BB on human B cells has not been elucidated.

Accordingly, there is need to understand the functional role of 4-1BB, in conjunction with binding by its ligand (4-1BBL or CD137L) and antibody (anti-4-1BB or anti-CD137 Ab), on human B cells to help develop treatment of cancers and diseases associated with 4-1BB expression.

### SUMMARY OF THE INVENTION

The present invention provides methods for treating cancers and diseases associated with 4-1BB expression, and methods for the diagnosis and tracking of such conditions.

According to a first embodiment, the present invention is directed to a method for inducing proliferation of B lymphocytes, comprising contacting B lymphocytes with an effective amount of 4-1BB ligand, thereby inducing proliferation of B lymphocytes.

According to a second embodiment, the present invention is directed to a method for activating B lymphocytes, comprising contacting B lymphocytes with an effective amount of 4-1BB ligand, thereby activating B lymphocytes.

According to a third embodiment, the present invention is directed to a method for promoting survival of B lymphocytes, comprising contacting B lymphocytes with an effective amount of 4-1BB ligand, thereby promoting survival of B lymphocytes.

According to a fourth embodiment, the present invention is directed to a method for inhibiting proliferation of B lymphocytes, comprising contacting B lymphocytes with an effective amount of an anti-4-1BB antibody, thereby inhibiting proliferation of B lymphocytes.

According to a fifth embodiment, the present invention is directed to a method for inhibiting activation of B lymphocytes, comprising contacting B lymphocytes with an effective amount of an anti-4-1BB antibody, thereby inhibiting activation of B lymphocytes.

According to a sixth embodiment, the present invention is directed to a method for inducing death of B lymphocytes, comprising contacting B lymphocytes with an effective amount of an anti-4-1BB antibody, thereby inducing death of B lymphocytes.

According to a seventh embodiment, the present invention is directed to a method for inducing proliferation of B lymphocytes in a mammal, comprising administering to a mammal a pharmaceutical composition that comprises a therapeutically effective amount of 4-1BB ligand and a carrier, thereby inducing proliferation of B lymphocytes in a mammal.

According to an eight embodiment, the present invention is directed to a method for activating B lymphocytes in a mammal, comprising administering to a mammal a pharmaceutical composition that comprises a therapeutically effective amount of 4-1BB ligand and a carrier, thereby activating B lymphocytes in a mammal.

According to a ninth embodiment, the present invention is directed to a method for promoting survival of B lymphocytes in a mammal, comprising administering to a mammal a pharmaceutical composition that comprises a therapeutically effective amount of 4-1BB ligand and a carrier, thereby promoting survival of B lymphocytes in a mammal.

According to a tenth embodiment, the present invention is directed to a method for inhibiting proliferation of B lymphocytes in a mammal, comprising administering to a mammal a pharmaceutical composition that comprises a therapeutically effective amount of anti-4-1BB antibody and a carrier, thereby inhibiting proliferation of B lymphocytes in a mammal.

According to an eleventh embodiment, the present invention is directed to a method for inhibiting activation of B lymphocytes in a mammal, comprising administering to a mammal a pharmaceutical composition that comprises a therapeutically effective amount of anti-4-1BB antibody and a carrier, thereby inhibiting activation of B lymphocytes in a mammal.

According to a twelfth embodiment, the present invention is directed to a method for inducing death of B lymphocytes in a mammal, comprising administering to a mammal a pharmaceutical composition that comprises a therapeutically effective amount of anti-4-1BB antibody and a carrier, thereby inducing death of B lymphocytes in a mammal.

In each of the above-mentioned embodiments, the mammal is a human.

According to the thirteenth embodiment, the present invention is directed to a method for treating a patient suffering from a B lymphocyte malignancy, comprising administering to a patient suffering from a B lymphocyte malignancy a pharmaceutical composition comprising a therapeutically effective amount of anti-4-1BB antibody and a carrier, thereby treating a patient suffering from a B lymphocyte malignancy.

According to the fourteenth embodiment, the present invention is directed to a method for treating a patient suffering from a B lymphocyte malignancy, comprising administering to a patient suffering from a B lymphocyte malignancy a pharmaceutical composition comprising a therapeutically effective amount of 4-1BB ligand and a carrier, thereby treating a patient suffering from a B lymphocyte malignancy.

In the thirteenth and fourteenth embodiments, a radioimmunotherapeutic agent or a chemotherapeutic agent may be administered prior to, concurrently with, or after administration of the pharmaceutical composition.

In the thirteenth and fourteenth embodiments, the B lymphocyte malignancy is selected from the group consisting of follicular lymphoma, endemic Burkitt's lymphoma, sporadic Burkitt's lymphoma, non-Burkitt's lymphoma, mucosa-associated lymphoid tissue MALT/MALToma (extranodal), monocytoid B-cell lymphoma (nodal), splenic lymphoma with villous lymphocytes, mantle cell lymphoma, diffuse large cell lymphoma, diffuse mixed large cell lymphoma, immunoblastic lymphoma, primary mediastinal B-cell lymphoma, pulmonary B cell angiocentric lymphoma, and small lymphocytic lymphoma.

According to the fifteenth embodiment, the present invention is directed to a method for treating a patient suffering from a disease associated with 4-1BB expression, comprising administering to a patient suffering from a disease associated with 4-1BB expression a pharmaceutical composition comprising a therapeutically effective amount of anti-4-1BB antibody and a carrier, thereby treating a patient suffering from a disease associated with 4-1BB expression.

According to the sixteenth embodiment, the present invention is directed to a method for treating a patient suffering from a disease associated with 4-1BB expression, comprising administering to a patient suffering from a disease associated with 4-1BB expression a pharmaceutical composition comprising a therapeutically effective amount of 4-1BB ligand and a carrier, thereby treating a patient suffering from a disease associated with 4-1BB expression.

In the fifteenth and sixteenth embodiments, the treatment is a reduction of 4-1BB expression in the patient. In addition, a radioimmunotherapeutic agent or a chemotherapeutic agent may be administered prior to, concurrently with, or after administration of the pharmaceutical composition. Furthermore, the disease associated with 4-1BB expression may be a cancer, a B lymphocyte malignancy, an autoimmune disease, asthma, an allergy, or a chronic graft-versus-host disease. The B lymphocyte malignancy is selected from the group consisting of follicular lymphoma, endemic Burkitt's lymphoma, sporadic Burkitt's lymphoma, non-Burkitt's lymphoma, mucosa-associated lymphoid tissue MALT/MALToma (extranodal), monocytoid B-cell lymphoma (nodal), splenic lymphoma with villous lymphocytes, mantle cell lymphoma, diffuse large cell lymphoma, diffuse mixed large cell lymphoma, immunoblastic lymphoma, primary mediastinal B-cell lymphoma, pulmonary B cell angiocentric lymphoma, and small lymphocytic lymphoma. In addition, the autoimmune disease is selected from the group consisting of rheumatoid arthritis (RA), systemic lupus erythematosus (SLE), arteriosclerosis, arthritis, Crohn's disease, Hashimoto's thyroiditis, Addison's disease, juvenile diabetes, diabetes, Grave's disease, myasthenia gravis, Reiter's syndrome, and multiple sclerosis.

According to the seventeenth embodiment, the present invention is directed to a method for inducing death of B lymphocytes in a patient undergoing a disease treatment, comprising administering to a patient undergoing a disease treatment a pharmaceutical composition comprising a therapeutically effective amount of anti-4-1BB antibody and a carrier, thereby inducing death of B lymphocytes in a patient undergoing a disease treatment.

According to the eighteenth embodiment, the present invention is directed to a method of promoting survival of B lymphocytes in a patient undergoing a disease treatment, comprising administering to a patient undergoing a disease treatment a pharmaceutical composition comprising a therapeutically effective amount of 4-1BB ligand and a carrier, thereby promoting survival of B lymphocytes in a patient undergoing a disease treatment.

In the seventeenth and eighteenth embodiments, the treatment may be radiation therapy or chemotherapy, wherein a radioimmunotherapeutic agent or a chemotherapeutic agent is administered prior to, concurrently with, or after administration of the pharmaceutical composition.

According to the nineteenth embodiment, the present invention is directed to a method for enhancing innate and/or adaptive immunity of a patient suffering from a B lymphocyte malignancy, comprising administering to a patient suffering from a B lymphocyte malignancy a pharmaceutical composition comprising a therapeutically effective amount of anti-4-1BB antibody and a carrier, thereby enhancing innate and/or adaptive immunity of a patient suffering from a B lymphocyte malignancy.

According to the twentieth embodiment, the present invention is directed to a method for enhancing innate and/or adaptive immunity of a patient suffering from a B lymphocyte malignancy, comprising administering to a patient suffering from a B lymphocyte malignancy a pharmaceutical composition comprising a therapeutically effective amount of 4-1BB ligand and a carrier, thereby enhancing innate and/or adaptive immunity of a patient suffering from a B lymphocyte malignancy.

In the nineteenth and twentieth embodiments, a radioimmunotherapeutic agent or a chemotherapeutic agent may be administered prior to, concurrently with, or after administration of the pharmaceutical composition. The B lymphocyte malignancy is selected from the group consisting of follicular lymphoma, endemic Burkitt's lymphoma, sporadic Burkitt's lymphoma, non-Burkitt's lymphoma, mucosa-associated lymphoid tissue MALT/MALToma (extranodal), monocytoid B-cell lymphoma (nodal), splenic lymphoma with villous lymphocytes, mantle cell lymphoma, diffuse large cell lymphoma, diffuse mixed large cell lymphoma, immunoblastic lymphoma, primary mediastinal B-cell lymphoma, pulmonary B cell angiocentric lymphoma, and small lymphocytic lymphoma.

According to the twenty-first embodiment, the present invention is directed to a method for enhancing innate and/or adaptive immunity of a patient suffering from a disorder associated with 4-1BB expression, comprising administering to a patient suffering from a disorder associated with 4-1BB expression a pharmaceutical composition comprising a therapeutically effective amount of anti-4-1BB antibody and a carrier, thereby enhancing innate and/or adaptive immunity of a patient suffering from a disorder associated with 4-1BB expression.

According to the twenty-second embodiment, the present invention is directed to a method for enhancing innate and/or adaptive immunity of a patient suffering from a disorder associated with 4-1BB expression, comprising administering to a patient suffering from a disorder associated with 4-1BB expression a pharmaceutical composition comprising a therapeutically effective amount of 4-1BB ligand and a carrier, thereby enhancing innate and/or adaptive immunity of a patient suffering from a disorder associated with 4-1BB expression.

In the twenty-first and twenty-second embodiments, a radioimmunotherapeutic agent or a chemotherapeutic agent may be administered prior to, concurrently with, or after administration of the pharmaceutical composition. In addition, the disorder associated with 4-1BB expression is a cancer, a B lymphocyte malignancy, an autoimmune disease, asthma, an allergy, or a chronic graft-versus-host disease. The B lymphocyte malignancy is selected from the group consisting of follicular lymphoma, endemic Burkitt's lymphoma, sporadic Burkitt's lymphoma, non-Burkitt's lymphoma, mucosa-associated lymphoid tissue MALT/MALToma (extranodal), monocytoid B-cell lymphoma (nodal), splenic lymphoma with villous lymphocytes, mantle cell lymphoma, diffuse large cell lymphoma, diffuse mixed large cell lymphoma, immunoblastic lymphoma, primary mediastinal B-cell lymphoma, pulmonary B cell angiocentric lymphoma, and small lymphocytic lymphoma. The autoimmune disease is selected from the group consisting of rheumatoid arthritis (RA), systemic lupus erythematosus (SLE), arteriosclerosis, arthritis, Crohn's disease, Hashimoto's thyroiditis, Addison's disease, juvenile diabetes, diabetes, Grave's disease, myasthenia gravis, Reiter's syndrome, and multiple sclerosis.

According to the twenty-third embodiment, the present invention is directed to a method for determining the responsiveness of a patient suffering from a disease to a therapy, comprising:

(a) determining the amount of 4-1BB and/or 4-1BB ligand present in a biological sample of a patient after receiving a therapy, and

(b) comparing the amount of 4-1BB and/or 4-1BB ligand determined in (a) to a control value obtained from a biological sample of said patient prior to receiving the therapy, wherein the result of the comparison of (b) provides a determination of the responsiveness of the patient suffering from the disease to the therapy.

In this embodiment, the disease is associated with 4-1BB expression and selected from the group consisting of cancer, B lymphocyte malignancy, autoimmune disease, asthma, allergy and chronic graft-versus-host disease. In addition, the B lymphocyte malignancy is selected from the group consisting of follicular lymphoma, endemic Burkitt's lymphoma, sporadic Burkitt's lymphoma, non-Burkitt's lymphoma, mucosa-associated lymphoid tissue MALT/MALToma (extranodal), monocytoid B-cell lymphoma (nodal), splenic lymphoma with villous lymphocytes, mantle cell lymphoma, diffuse large cell lymphoma, diffuse mixed large cell lymphoma, immunoblastic lymphoma, primary mediastinal B-cell lymphoma, pulmonary B cell angiocentric lymphoma, and small lymphocytic lymphoma. Furthermore, the autoimmune disease is selected from the group consisting of rheumatoid arthritis (RA), systemic lupus erythematosus (SLE), arteriosclerosis, arthritis, Crohn's disease, Hashimoto's thyroiditis, Addison's disease, juvenile diabetes, diabetes, Grave's disease, myasthenia gravis, Reiter's syndrome, and multiple sclerosis.

According to the twenty-fourth embodiment, the present invention is directed to a method for evaluating the progression of a disease in a patient, comprising:

(a) determining the amount of 4-1BB and/or 4-1BB ligand present in a biological sample of a patient, and

(b) comparing the amount of 4-1BB and/or 4-1BB ligand determined in (a) to a control value obtained from a biological sample of said patient at a date earlier than the date upon which the biological sample of (a) was obtained, wherein the result of the comparison of (b) provides an evaluation of the progression of the disease in the patient.

In this embodiment, the disease is associated with 4-1BB expression and selected from the group consisting of cancer, B lymphocyte malignancy, autoimmune disease, asthma, allergy and chronic graft-versus-host disease. The B lymphocyte malignancy is selected from the group consisting of follicular lymphoma, endemic Burkitt's lymphoma, sporadic Burkitt's lymphoma, non-Burkitt's lymphoma, mucosa-associated lymphoid tissue MALT/MALToma (extranodal), monocytoid B-cell lymphoma (nodal), splenic lymphoma with villous lymphocytes, mantle cell lymphoma, diffuse large cell lymphoma, diffuse mixed large cell lymphoma, immunoblastic lymphoma, primary mediastinal B-cell lymphoma, pulmonary B cell angiocentric lymphoma, and small lymphocytic lymphoma. The autoimmune disease is selected from the group consisting of rheumatoid arthritis (RA), systemic lupus erythematosus (SLE), arteriosclerosis, arthritis, Crohn's disease, Hashimoto's thyroiditis, Addison's disease, juvenile diabetes, diabetes, Grave's disease, myasthenia gravis, Reiter's syndrome, and multiple sclerosis.

According to the twenty-fifth embodiment, the present invention is directed to a method for diagnosing a disease associated with aberrant 4-1BB expression, comprising:

(a) determining the amount of 4-1BB and/or 4-1BB ligand present in a biological sample of a subject; and

(b) comparing the amount of 4-1BB and/or 4-1BB ligand determined in (a) to a control value obtained from biological samples of a population of subjects lacking aberrant 4-1BB expression, thereby diagnosing a disease associated with aberrant 4-1BB expression.

In this embodiment, the disease is associated with aberrant 4-1BB expression is a cancer, a B lymphocyte malignancy, an autoimmune disease, asthma, an allergy, or a chronic graft-versus-host disease. In addition, the B lymphocyte malignancy is selected from the group consisting of follicular lymphoma, endemic Burkitt's lymphoma, sporadic Burkitt's lymphoma, non-Burkitt's lymphoma, mucosa-associated lymphoid tissue MALT/MALToma (extranodal), monocytoid B-cell lymphoma (nodal), splenic lymphoma with villous lymphocytes, mantle cell lymphoma, diffuse large cell lymphoma, diffuse mixed large cell lymphoma, immunoblastic lymphoma, primary mediastinal B-cell lymphoma, pulmonary B cell angiocentric lymphoma, and small lymphocytic lymphoma. Furthermore, the autoimmune disease is selected from the group consisting of rheumatoid arthritis (RA), systemic lupus erythematosus (SLE), arteriosclerosis, arthritis, Crohn's disease, Hashimoto's thyroiditis, Addison's disease, juvenile diabetes, diabetes, Grave's disease, myasthenia gravis, Reiter's syndrome, and multiple sclerosis.

It is contemplated that any method or composition described herein can be implemented with respect to any other method or composition described herein. Moreover, it is clearly contemplated that embodiments may be combined with one another, to the extent that they are compatible.

Other features and advantages of the present invention are apparent in the detailed description that follows. It should be understood, however, that the detailed description and the specific examples, while indicating embodiments of the present invention, are given by way of illustration only, not limitation. Various changes and modifications within the scope of the invention will become apparent to those skilled in the art from the detailed description.

### BRIEF DESCRIPTION OF THE DRAWINGS

Details of the embodiments of the present invention are illustrated in the appended drawings.

FIGS. 1A-E shows the induction of 4-1BB (CD137) on human B cells following antigen exposure while expression levels are enhanced by cognate interactions between CD40 and CD40 ligand. FIG. 1A: Human B cells up-regulate 4-1BB (CD137) in the presence of pokeweed mitogen (PWM). Histograms indicate percentage 4-1BB (CD137) expression on gated B (CD19⁺) cells in whole PBMC. Data are representative of at least 5 individual experiments. FIG. 1B: B cells require T cell contact in order to up-regulate CD137 during PWM stimulation. Histograms indicate percentage 4-1BB (CD137) expression on gated B (CD19⁺) cells cultured in the presence (no transwell) or absence (transwell) of T cells. Data are representative of 4 individual experiments. FIG. 1C: Anti-CD40L (solid squares) blocks 4-1BB (CD137) expression on PWM activated B cells in PBMC and B/T cell co-cultures in a dose-dependent fashion. Data are presented as mean±SD and are representative of 5 individual experiments. FIG. 1D: 4-1BB (CD137) is induced on human B cells upon BCR stimulation with enhancement of cell surface expression levels following CD40 ligation with agonistic anti-CD40 antibody. Histograms indicate percentage 4-1BB (CD137) expression on gated B (CD19⁺) cells. FIG. 1E: RT-PCR confirmed the up-regulation of 4-1BB (CD137) mRNA in anti-Ig/anti-CD40 stimulated B cells after 12 hours of culture. Data are representative of 3 individual experiments. *P<0.05 and **P<0.0001

FIGS. 2A-B shows that cytokines can provide a second level of regulatory control for the expression of B cell associated 4-1BB (CD137). FIG. 2A: Purified B (CD19⁺) cells were anti-Ig/anti-CD40 stimulated in the presence of no cytokine, IL-2, IL-4, IL-6, IL-10, IL-15, IL-21, TNFα or IFN-γ. After 3 days of culture, B cells were harvested and assessed for 4-1BB (CD137) expression by flow cytometry. 4-1BB (CD137) expression levels are significantly enhanced (P<0.0001) in the presence of IFN-γ. In contrast, 4-1BB (CD137) expression levels are significantly reduced in the presence of IL-4 (P<0.0001) and IL-10 (P=0.0188). FIG. 2B: CD40 ligation and/or IFN-γ enhance 4-1BB (CD137) expression levels on human B cells but primary B cell receptor stimulation is required. Histograms indicate percentage of 4-1BB (CD137) expression on B (CD19⁺) cells. Data are representative of at least 5 individual experiments.

FIGS. 3A-B shows the preferential expression of 4-1BB (CD137) on activated B cells of naïve origin. FIG. 3A: Purified human B cells were anti-Ig/anti-CD40 activated. After 3 days, B cells were harvested and 4-1BB (CD137) expressing (CD19⁺CD137⁺) B cells and non-CD137 expressing (CD19⁺CD137⁻) B cells were assessed for cell surface phenotype by flow cytometry. Histograms show percentage surface expression of indicated marker on CD137⁺ B cells (dotted line) and CD137⁻ B cells (solid line). Filled peaks represent isotype controls. Data are representative of 5 individual experiments. FIG. 3B: Purified human B cells were separated into naïve (CD19⁺CD27⁻) and memory (CD19⁺CD27⁺) B cells and subsequently anti-Ig/anti-CD40 stimulated. CD137 surface expression was determined at indicated time-points. Data shown are representative of 5 individual experiments. *P<0.05.

FIGS. 4A-C shows that 4-1BB (CD137) expression is associated with enhanced B cell activation. FIGS. 4A and 4B: High levels of CD137 are expressed on divided human B cells. Unseparated (bulk; CD19⁺) B cells, naïve (CD19⁺CD27⁻) and memory (CD19⁺CD27⁺) B cells were CFSE labeled and stimulated with anti-Ig/anti-CD40. After 4 days, cells were harvested and analyzed for CFSE dilution and 4-1BB (CD137) expression by flow cytometry. Based on CFSE dilution, divided and non-divided B cell subsets were gated and analyzed for CD137 surface expression. Histograms indicate percentage of 4-1BB (CD137) expression on indicated subset of divided (FIG. 4A) and non-divided (FIG. 4B) B cells. Data are representative of at least 5 individual experiments. FIG. 4C: 4-1BB (CD137) expression is associated with cell cycle progression in anti-Ig/anti-CD40 stimulated human B cells. Anti-Ig/anti-CD40 stimulated B cells were cultured for 3 days and pulsed with BrdU for 4 hours. Cell cycle status of 4-1BB (CD137) expressing (CD19⁺CD137⁺) and non-CD137 expressing (CD19⁺CD137⁻) B cells was confirmed by flow cytometry using anti-BrdU and 7-AAD. Data are representative of 3 individual experiments.

FIGS. 5A-D shows that 4-1BB (CD137) ligation can stimulate B cell proliferation. FIG. 5A: Purified B cells were stimulated with anti-Ig, anti-Ig/IFN-γ or anti-Ig/anti-CD40 in the presence of various numbers of irradiated P815/mock (filled diamonds) or P815/CD137L-expressing cells (filled squares) as indicated in FIG. 5A. After 3 days (anti-Ig, anti-Ig/IFN-γ) or 4 days (anti-Ig/antiCD40) of culture, cellular activation was measured by thymidine (³H-TdR) incorporation. Data are presented as mean±SD of triplicate wells and are representative of 5 experiments. FIG. 5B: The addition of soluble CD137 (sCD137) protein to cultures with anti-Ig activated B cells abrogates the observed effect; P815-mock (grey bars) or P815-CD137L (black bars). Data are presented as mean±SD of triplicate wells and are representative of 4 individual experiments. FIGS. 5C and 5D: Anti-Ig stimulated B cells exhibit greater CFSE dilution (FIG. 5C) and have more B cells in the S phase of the cell cycle (FIG. 5D) in the presence of irradiated P815-CD137L cells in comparison to P815-mock controls. Data are representative of 4 individual experiments. *P<0.05, **P<0.001.

FIGS. 6A-D shows that 4-1BB (CD137) ligation can enhance B cell survival. FIG. 6A: Purified B cells were stimulated with anti-Ig, anti-Ig/IFN-γ and anti-Ig/anti-CD40 in the presence of P815-mock or P815-CD137L cells. The percentage of B cell survival (Annexin V⁻/7-AAD⁻) was assessed by flow cytometry at culture day 4 (D4) and day 6 (D6). FIG. 6B: The absolute number of surviving B cells in these cultures was assessed by flow cytometry using accucount beads. FIGS. 6C and 6D: 4-1BB (CD137) co-stimulation promotes B cell survival after treatment with Doxorubicin (FIG. 6C) or growth factor deprivation (FIG. 6D). In each figure, bar graphs represent B cells survival in cultures with P815-mock (white bars) or P815-CD137L (grey bars). Data are shown as mean±SD and represent at least 4 (a,b) and 3 (c,d) individual experiments. *P<0.05.

FIG. 7 shows that 4-1BB (CD137) ligation is associated with the up-regulation of anti-apoptotic proteins. Purified (CD19⁺) B cells were anti-Ig, anti-Ig/IFN-γ or anti-Ig/anti-CD40 stimulated in the presence of irradiated P815-mock or P815-CD137L. After 3 days of culture, cells were harvested and CD19⁺ B cell purity was enhanced using anti-CD19 microbeads. Purified B cells were used to prepare protein lysates and protein expression of bcl-2, bcl-xL and mcl-1 was determined by western blot analysis. β-actin served as an internal control and data are representative of 3 individual experiments.

FIGS. 8A-B: CD137 costimulation on anti-BCR activated B cells enhance the production of IgM and IgG in healthy donor. Purified B cells from peripheral blood of healthy donor were activated with anti-Ig in the presence of mock or 4-1BBL transfectants for 3 days. Then cells were incubated with cytokines for 10 days. Total IgM (FIG. 8A) and IgG (FIG. 8A) levels in the culture supernatant were determined by ELISA.

FIG. 9: Peripheral blood B cells from patients with active RA express higher level of CD137 upon activation. PBMC from different patient groups were activated with anti-Ig only (FIG. 9A) or anti-Ig/anti-CD40 (FIG. 9B) for 24 hrs. CD137 expression on B cells was determined by multi-color FACS analysis. In comparison with healthy control group, patients with active RA upregulate higher level of CD137 expression on PB B cells upon activation with anti-BCR in the absence of presence of anti-CD40 stimulation.

### DETAILED DESCRIPTION OF THE INVENTION

The present invention is based on a novel finding that 4-1BB is a survival and proliferative factor of human B lymphocytes and that anti-4-1BB (anti-CD137) antibodies inhibit proliferation and activation, and induce death of human B lymphocytes.

The 4-1BB receptor is distinct from other TNF receptor family members that regulate B cell proliferation and differentiation, such as CD40 and BAFF-Receptor (BAFF-R) (Refs. 38, 43 and 44). CD40 and BAFF-R are constitutively expressed on B cells whereas the upregulation of 4-1BB is strictly dependent on the presence of anti-BCR stimulation. Similar to BAFF-R, 4-1BB co-stimulates the proliferation of B cells in the presence of anti-Ig. In contrast, CD40 stimulates B cell proliferation in the absence of anti-Ig. Therefore, it is likely that CD40, 4-1BB and BAFF-R fulfill distinct functions in vivo.

The data presented herein below suggest that 4-1BB (4-1BB) blockade may be effective for the treatment of cancers and diseases wherein B cells play a role in their pathogenesis. The data also suggest that 4-1BB (4-1BB) blockade may be relevant for the treatment of B cell malignancies and potentially other malignancies associated with 4-1BB expression. It is also likely that blocking antibodies against 4-1BB can be used in combination with radioimmunotherapy and/or chemotherapy for the treatment of cancers or diseases that are associated with 4-1BB expression.

Thus, the overall findings obtained herein have important implications for the clinical translation of 4-1BB-based immunotherapeutic strategies. The disparity of 4-1BB expression and function between human and murine B cells challenges the use of murine-based disease models for the evaluation of 4-1BB-mediated immune regulation, and targeting the 4-1BB pathway with therapeutic intent may have unanticipated consequences on human B cell function. In addition, evidence that 4-1BB ligation protects B cells from chemotherapy-induced apoptosis may be of both prognostic and therapeutic relevance when treating certain types of B cell lymphomas expressing 4-1BB (45). Furthermore, the fact that the expression of B cell-associated 4-1BB is regulated by many of the factors involved in the pathogenesis of rheumatoid arthritis (RA) and systemic lupus arthritis (SLE), e.g., enhanced CD40 expression and altered cytokine production, suggests that B cell-associated 4-1BB might be of functional import in these diseases (24, 25).

### Methods of Inducing Proliferation and Activation and Promoting Survival

Accordingly, the present invention is directed to various methods, including methods of inducing B lymphocyte proliferation, methods of activating B lymphocytes and methods of promoting survival of B lymphocytes. Each of these methods involves contacting B lymphocytes with an effective amount of 4-1BB ligand.

The application of each of these methods may be conducted in vitro, in vivo or ex vivo. In addition, such methods can be carried out in mammals, such as humans, through the administration to the mammals of a pharmaceutical composition that includes a therapeutically effective amount of 4-1BB ligand and a carrier.

### Methods of Inhibiting Proliferation and Activation and Inducing Death

The present invention is also directed to various methods, including methods of inhibiting B lymphocyte proliferation, methods of inhibiting activation of B lymphocytes and methods of inducing death of B lymphocytes. All of these methods involve contacting B lymphocytes with an effective amount of an anti-4-1BB antibody. In addition, all of these methods can be applied either in vitro, in vivo or ex vivo. These methods can be conducted in mammals, such as humans, through the administration to the mammals of a pharmaceutical composition that includes a therapeutically effective amount of anti-4-1BB antibody and a carrier.

### Methods of Treatment Using Anti-4-1BB Antibody or 4-1BB Ligand

The present invention is also directed to methods of treating a patient suffering from a B lymphocyte malignancy utilizing a pharmaceutical composition comprising a therapeutically effective amount of anti-4-1BB antibody and a carrier.

In an alternative embodiment, a pharmaceutical composition comprising a therapeutically effective amount of 4-1BB ligand and a carrier can be used in this method. The skilled artisan will understand that the identity of the active agent will depend on the nature of the B lymphocyte malignancy.

Such methods of treatment can be performed in conjunction with a radioimmunotherapeutic agent or a chemotherapeutic agent either prior to, concurrently with, or after administration of the pharmaceutical composition.

The present invention is further directed to methods for treating a patient suffering from a disease associated with 4-1BB expression using a pharmaceutical composition that includes either an anti-4-1BB antibody and a carrier, or a 4-1BB ligand and a carrier. Administration of such pharmaceutical composition to the patient suffering from a disease associated with 4-1BB expression would thereby result to the reduction of 4-1BB expression in the patient.

In one variation, the method further includes the administration of a radioimmunotherapeutic agent or a chemotherapeutic agent that can be administered prior to, concurrently with, or after administration of the pharmaceutical composition.

### Methods of Inducing Death or Promoting Survival of B Lymphocytes

The present invention is further directed to a method of inducing death of B lymphocytes in a patient undergoing a disease treatment, wherein the treatment is radiation therapy or chemotherapy. The method encompasses administering to the patient a pharmaceutical composition comprising a therapeutically effective amount of anti-4-1BB antibody and a carrier, thereby inducing death of B lymphocytes in the patient undergoing such treatment.

In an alternative embodiment, the method may also be used to promote the survival of B lymphocytes in a patient undergoing a disease treatment, wherein the treatment is radiation therapy or chemotherapy. The method entails administering to the patient a pharmaceutical composition comprising a therapeutically effective amount of 4-1BB ligand and a carrier, thereby promoting survival of B lymphocytes in the patient undergoing such treatment.

In one variation, the above-mentioned methods may include the administration of a radioimmunotherapeutic agent or a chemotherapeutic agent that can be administered prior to, concurrently with, or after administration of the pharmaceutical composition.

Methods of Enhancing Innate and/or Adaptive Immunity

The present invention is also directed to methods of enhancing innate and/or adaptive immunity of a patient suffering from a B lymphocyte malignancy or a disorder associated with 4-1BB expression. These methods can be carried out by administering to patients suffering from either a B lymphocyte malignancy or a disorder associated with 4-1BB expression a pharmaceutical composition that encompasses a therapeutically effective amount of anti-4-1BB antibody and a carrier, thereby enhancing innate and/or adaptive immunity of such patients.

Instead of administering with the anti-4-1BB antibody, the above-mentioned methods can be alternatively carried out by using a pharmaceutical composition comprising a therapeutically effective amount of 4-1BB ligand and a carrier, thereby enhancing innate and/or adaptive immunity of the patient suffering from either a B lymphocyte malignancy or a disorder associated with 4-1BB expression.

As one variation, the above-mentioned methods may include the administration of a radioimmunotherapeutic agent or a chemotherapeutic agent that can be administered prior to, concurrently with, or after administration of the pharmaceutical composition.

Disorders associated with 4-1BB expression may include a cancer, a B lymphocyte malignancy, an autoimmune disease, asthma, an allergy, or a chronic graft-versus-host disease.

### Methods for Determining the Responsiveness of a Disease

The present invention further provides a method for determining the responsiveness of a patient suffering from a disease to a therapy. The method encompasses: (a) determining the amount of 4-1BB and/or 4-1BB ligand present in a biological sample of a patient after receiving a therapy, and (b) comparing the amount of 4-1BB and/or 4-1BB ligand determined in (a) to a control value obtained from a biological sample of the patient prior to receiving the therapy, wherein the result of the comparison of (b) provides a determination of the responsiveness of the patient suffering from the disease to the therapy.

In one embodiment, the disease is associated with 4-1BB expression and may include a cancer, a B lymphocyte malignancy, an autoimmune disease, asthma, an allergy, or a chronic graft-versus-host disease.

Such information can be useful and important in monitoring the effectiveness of a course of therapy and in determining means for further treatment.

### Methods for Evaluating the Progression of a Disease

The present invention is further directed to a method for evaluating the progression of a disease in a patient which includes: (a) determining the amount of 4-1BB and/or 4-1BB ligand present in a biological sample of a patient, and (b) comparing the amount of 4-1BB and/or 4-1BB ligand determined in (a) to a control value obtained from a biological sample of the patient at a date earlier than the date upon which the biological sample of (a) was obtained, and wherein the result of the comparison of (b) provides an evaluation of the progression of the disease in the patient.

In one embodiment, the disease may be cancer, B lymphocyte malignancy, autoimmune disease, asthma, allergy or chronic graft-versus-host disease.

Such information can be useful and important in monitoring the effectiveness of a course of therapy and in determining means for further treatment.

### Methods for Diagnosing a Disease

The present invention is also directed to a method for diagnosing a disease associated with aberrant 4-1BB expression which includes the following: (a) determining the amount of 4-1BB and/or 4-1BB ligand present in a biological sample of a subject; and (b) comparing the amount of 4-1BB and/or 4-1BB ligand determined in (a) to a control value obtained from biological samples of a population of subjects lacking aberrant 4-1BB expression, thereby diagnosing a disease associated with aberrant 4-1BB expression.

In one embodiment, the disease that is associated with aberrant 4-1BB expression may include cancer, B lymphocyte malignancy, autoimmune disease, asthma, allergy and chronic graft-versus-host disease.

The skilled artisan will understand that the term “mammal” includes human and non-human primates. A subject may be a patient in need of a treatment for a disease defined herein or maybe an individual with no symptoms or disease. A subject can also refer to a cancer patient who is undergoing anti-cancer therapy-induced cell death, before, during or after anti-cancer treatment.

The term “inducing proliferation of B lymphocytes,” as used herein, may refer to an increase in the numbers of B lymphocytes, as determined, for example, by an increase in the expression of cell markers associated with cell proliferation, an increase in the number of rounds of cell cycle progression as indicated by flow cytometry, an elevated DNA uptake as measured by thymidine incorporation, and/or a simple increase in the number of B lymphocytes.

The term “inhibiting proliferation of B lymphocytes,” as used herein, may refer to a slowing and/or preventing the growth and division of B lymphocytes, as determined, for example, by a decreased expression of cell markers associated with cell proliferation, a reduction of DNA uptake as measured by thymidine incorporation and/or reduction of cell division as measured by flow cytometry, and/or a simple decrease in the number of B lymphocytes.

The term “promoting survival of B lymphocytes,” as used herein, refers to maintaining the life and growth of B lymphocytes in a certain population, as determined, for example, by the upregulation of expression of anti-apoptotic proteins or no consistent changes of the expression of the pro-apoptotic proteins.

The term “inducing death of B lymphocytes,” as used herein, may refer to increasing the number of B lymphocytes undergoing cell death or apoptosis or the rate by which the B lymphocytes undergo death in a given population, as detected, for example, by DNA fragmentation, formation of membrane vesicles, binding of annexin V, cell shrinkage, etc.

Specific or non-specific binding of anti-4-1BB antibody with an antigen of interest on the surface of the B cell lymphocyte can lead to reduction or prevention of a humoral response by the B lymphocytes, which, in turn, may lead to inhibition of cell proliferation, inhibition of activation of B lymphocytes or induction of death of B lymphocytes.

Innate immunity refers to an early system of defense that depends on invariant receptors recognizing common features of pathogens. The innate immune system provides barriers and mechanisms to inhibit foreign substances, in particular through the action of macrophages and neutrophils. The inflammatory response is considered part of innate immunity. The innate immune system is involved in initiating adaptive immune responses and removing pathogens that have been targeted by an adaptive immune response. However, innate immunity can be evaded or overcome by many pathogens, and does not lead to immunological memory.

Adaptive immune response or “adaptive immunity” is the response of antigen-specific lymphocytes to antigen, including the development of immunological memory. Adaptive immune responses are generated by clonal selection of lymphocytes bearing antigen-specific receptors and are distinct from innate and non-adaptive phases of immunity, which are not mediated by clonal selection of antigen-specific lymphocytes. Adaptive immunity, as used herein, includes cellular and humoral immunity. Immune recognition by the adaptive immune system is mediated by antigen receptors.

The term “enhancing innate and/or adaptive immunity,” as used herein, refers to the stimulation, activation or augmentation of the innate and/or adaptive immune system of a mammal, a human or a patient suffering from cancer or a disease associated with 4-1BB expression, which results to the production of constituents of cellular and/or humoral immune responses.

The term “suffering from a disease,” as used herein, may refer to a mammal, patient or subject who is experiencing, undergoing or showing signs and symptoms of and/or pain due to a medical ailment or condition associated with cancer or a disease associated with 4-1BB expression.

The term “aberrant 4-1BB expression,” as used herein, refers to a change or deviation from the normal level or amount of 4-1BB and/or 4-1BB ligand in a control population of subjects. The change or deviation may involve a slight, higher or highest increase or decrease of the amount of 4-1BB and/or 4-1BB ligand compared to the control population.

The terms “control population” and “a population of subjects lacking aberrant 4-1BB expression,” shall be taken to mean a population of subjects having a normal level or amount of 4-1BB expression, having no cancer or disease associated with 4-1BB expression, and/or being asymptomatic with respect to any symptoms associated with cancer or disease associated with 4-1BB expression.

The term “chemotherapeutic agent,” as referred herein, is a chemical compound that prevents the development, maturation or spread of neoplastic cells, and that acts directly on the tumor cell, e.g., by cytostatic or cytotoxic effects, and not indirectly through mechanisms such as biological response modification. Suitable chemotherapeutic agents are preferably natural or synthetic chemical compounds, but biological molecules, such as proteins, polypeptides, etc. are not expressively excluded.

Examples of chemotherapeutic agents include alkylating agents, for example, nitrogen mustards, ethyleneimine compounds, alkyl sulphonates and other compounds with an alkylating action such as nitrosoureas, cisplatin and dacarbazine; antimetabolites, for example, folic acid, purine or pyrimidine antagonists; mitotic inhibitors, for example, vinca alkaloids and derivatives of podophyllotoxin; cytotoxic antibiotics and camptothecin derivatives. More particularly, chemotherapeutic agents or chemotherapy may include amifostine (ethyol), cisplatin, dacarbazine (DTIC), dactinomycin, mechlorethamine (nitrogen mustard), streptozocin, cyclophosphamide, carrnustine (BCNU), lomustine (CCNU), doxorubicin (adriamycin), doxorubicin lipo (doxil), gemcitabine (gemzar), daunorubicin, daunorubicin lipo (daunoxome), procarbazine, mitomycin, cytarabine, etoposide, methotrexate, 5-fluorouracil (5-FU), vinblastine, vincristine, bleomycin, paclitaxel (taxol), docetaxel (taxotere), aldesleukin, asparaginase, busulfan, carboplatin, cladribine, camptothecin, CPT-11, 10-hydroxy-7-ethyl-camptothecin (SN38), dacarbazine, floxuridine, fludarabine, hydroxyurea, ifosfamide, idarubicin, mesna, interferon alpha, interferon beta, irinotecan, mitoxantrone, topotecan, leuprolide, megestrol, melphalan, mercaptopurine, plicamycin, mitotane, pegaspargase, pentostatin, pipobroman, plicamycin, streptozocin, tamoxifen, teniposide, testolactone, thioguanine, thiotepa, uracil mustard, vinorelbine, chlorambucil and combinations thereof.

Radioimmunotherapeutic (RIT) agents are used for the treatment of tumors and are well known in the art. Suitable RIT agents include any targeted radionuclide therapy with any therapeutically useful radionuclide attached, and which are attachable to or capturable by a tumor or tumor vasculature. Such useful RIT agents include a tumor-targeting or tumor vasculature-targeting ligand or molecule. The radionuclide can be attached directly to the targeting molecule or ligand, or by a chelating agent attached to or associated with the ligand. Alternatively, the ligand can include a chelating or radionuclide capturing group, and can be administered to the patient cold, to bind to a tumor or tumor vasculature in the patient. Upon subsequent administration of a radionuclide, the bound ligand can capture the radionuclide at the tumor site (pre-targeted radionuclide therapy). Targeting molecules or ligands include antibodies, antibody fragments, recombinant combinations of antibody fragments, peptides, or any other ligand that has a selective affinity for tumors or tumor vasculature.

Preferably, the RIT is a radionuclide-labeled chelating agent-ligand complex in which chelating agent is chemically bonded to a tumor-targeting molecule. Preferred tumor-targeting molecules include antibodies, such as monoclonal antibodies, or antibody fragments. More preferably, the tumor-targeting molecule is an anti-tumor antibody. Any anti-tumor antibody can be utilized. Preferably, the anti-tumor antibody targets tumor vasculature. Any radionuclide suitable for use in cancer radiotherapeutic methods can be utilized in the methods of the present invention. Suitable radionuclides include, without limitation, ¹³¹I, ¹⁷⁷Lu, ⁶⁷Cu, ⁶⁴Cu, ¹⁹⁶Re, and ⁹⁰Y.

A “biological sample” encompasses any of a variety of sample types obtainable from a mammal, a subject or a human and contains sufficient quantities of 4-1BB ligand and/or 4-1BB useful for a diagnostic or monitoring purpose. A biological sample encompasses blood and other liquid samples of biological origin, solid tissue samples, such as a biopsy specimen, or tissue cultures or cells derived therefrom and the progeny thereof. The term may also encompass a clinical sample, and further includes cells in cell culture, cell supernatants, cell lysates or extracts, serum, plasma, urine, amniotic fluid, biological fluids, and tissue samples.

As used herein, a “disease associated with 4-1BB (CD137) expression” may include cancer, B lymphocyte malignancy, autoimmune disease, asthma, allergy and chronic graft-versus-host disease.

A “B lymphocyte malignancy,” as used herein, may include follicular lymphoma, endemic Burkitt's lymphoma, sporadic Burkitt's lymphoma, non-Burkitt's lymphoma, mucosa-associated lymphoid tissue MALT/MALToma (extranodal), monocytoid B-cell lymphoma (nodal), splenic lymphoma with villous lymphocytes, mantle cell lymphoma, diffuse large cell lymphoma, diffuse mixed large cell lymphoma, immunoblastic lymphoma, primary mediastinal B-cell lymphoma, pulmonary B cell angiocentric lymphoma, and small lymphocytic lymphoma.

An “autoimmune disease,” as referred herein, includes any autoimmune disease wherein elimination or depletion or inhibition of the activity or proliferation of B cells is therapeutically beneficial. Such autoimmune diseases will include in particular T and B cell mediated autoimmune diseases Examples include rheumatoid arthritis (RA), systemic lupus erythematosus (SLE), arteriosclerosis, arthritis, Crohn's disease, Hashimoto's thyroiditis, Addison's disease, juvenile diabetes, diabetes, Grave's disease, myasthenia gravis, Reiter's syndrome and multiple sclerosis.

As used herein, the terms “cancer” and “tumor” are used interchangeably and refer to or describe the physiological condition in mammals in which a population of cells is characterized by unregulated cell growth. Examples of cancer include, but are not limited to, carcinoma, lymphoma, blastoma, sarcoma, and leukemia. More particular examples of such cancers include squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, vulval cancer, thyroid cancer, hepatic carcinoma and various types of head and neck cancers.

The terms “treating” or “treatment” refer to administering to a mammal, patient or subject a therapeutically effective amount of anti-4-1BB antibody or 4-1BB ligand so that the mammal, patient or subject has an improvement in a cancer or disease. The improvement is any improvement or remediation of the symptoms of the cancer or disease. The improvement may be an observable or measurable improvement. Thus, one of skill in the art realizes that a treatment may improve the disease condition, but may not be a complete cure for the disease. Specifically, improvements in patients with cancer may include tumor stabilization, tumor shrinkage, increased time to progression, increased survival or improvements in the quality of life. Improvements in patients with autoimmune disease may include improvement in laboratory values of inflammation, improvements in blood counts, improvements in rash, or improvements in the quality of life.

Preferably, treatment results in a measurable improvement where the improvement is a decrease of about 100%, 99%, 98%, 97%, 96%, 95%, 90%, 80%, 70%, 60%, 50%, 40%, 30%, 20%, 10%, 5% or 1% in the symptoms of the disease or condition and/or in the underlying physical basis of the disease or condition. The treatment may begin prior to, concurrent with, or after the onset of clinical symptoms of the disease or condition. Treatment may also comprise treating subjects at risk of developing a disease and/or condition.

The terms “preventing” and “prevention” as used herein refer to minimizing, reducing or suppressing: (i) the risk of developing a disease state or condition, (ii) parameters relating to a disease state or condition, and (iii) progression of a disease state or condition. As used herein, the prevention lasts at least one week, two weeks, three weeks, one month, two months, three, months, four months, five months, six months, seven months, eight months, nine months, ten months, eleven months, one year, two years, three years, four years, five years, six years, seven years or more, or indefinitely.

The terms “therapeutically effective amount” and “effective amount,” as used herein, refer to an amount that results in an improvement or remediation of a symptom of cancer or a disease.

An antibody for use in the present invention may bind non-specifically or specifically to 4-1BB. The term “anti-4-1BB antibody” may comprise a polyclonal or monoclonal anti-4-1BB antibody, chimeric anti-4-1BB antibody, and humanized anti-4-1BB antibody or epitope-binding fragments thereof such as the Fab and F(ab)₂ fragments. Furthermore, the skilled artisan will appreciate that the anti-4-1BB antibodies include antibodies with different affinities for the same epitope, as well as antibodies that recognize and bind different epitopes of 4-1BB, again with different affinities. The anti-4-1BB antibodies can differ in their glycosylation state and still be bioactive and differ in their effectiveness profile for their specific therapeutic applications. Accordingly, they can be either in aglycosylated form or as a glycosylated form of anti-4-1BB antibody.

Therapeutic mouse mAbs that require repeated administration for a full clinical effect are unsuitable for human use because the HAMA response neutralizes the antibody, clears it quickly from the circulation and, in the worst case, induces serious allergic hypersensitivity. Several strategies have been developed to replace most of the murine Ig sequences with human sequences, resulting in fewer side effects while retaining efficacy. The HAMA response may not be a serious problem with anti-4-1BB antibodies because of the potential inhibitory effects of anti-4-1BB antibodies on antibody production. Therefore, one strategy for developing a human therapeutic mAb is to replace the murine heavy chain (H) and light chain (L) constant regions (C_{H} and C_{L}, respectively) with human regions so that the resulting chimeric antibody is comprised mostly of human IgG protein sequence except for the antigen-binding domains. This is the strategy used for Rituxan™ (Rituximab anti-human CD20, Genentech), the first monoclonal antibody approved in the U.S. to treat non-Hodgkin lymphoma. By some estimates, providing therapeutic mAbs with human CH and CL sequences should eliminate approximately 90% of the immunogenicity of murine antibody proteins.

An alternative strategy for developing a clinical mAb product is to produce antibody in transgenic mice in which the entire native Ig repertoire has been replaced with human Ig genes. Such mice produce fully human antibody proteins. In this way a chimeric, humanized or fully human antibody is produced as one of several preferred embodiments of the current invention. However, both this antibody and a chimeric one would retain their effector function and would be useful in the treatment of cancer and cancerous lesions. The proposed chimeric antibody embodiment of the current invention retains the original murine variable (antigen-binding) sequences and hence should retain its binding and functional properties.

### Formulations

The 4-1BB (CD137) ligand (4-1BBL; CD137L) and anti-4-1BB (anti-CD137) antibody described herein can be formulated in a variety of useful formats for administration by a variety of routes. Concentrations of the 4-1BBL and anti-4-1BB antibody described will be such that a therapeutically effective dose of the 4-1BBL and anti-4-1BB antibody is included in the formulation, e.g., a pharmaceutical composition comprising a therapeutically effective dose of the 4-1BBL or anti-4-1BB antibody and a carrier. Determination of such a concentration would be readily apparent to those of ordinary skill in the art.

In one embodiment, the 4-1BBL and anti-4-1BB antibody of the present invention may be formulated, for example, for oral, sublingual, intranasal, intraocular, rectal, transdermal, mucosal, topical or parenteral administration. Parenteral modes of administration include, without limitation, intradermal, subcutaneous (s.c., s.q., sub-Q, Hypo), intramuscular (i.m.), intravenous (i.v.), intraperitoneal (i.p.), intra-arterial, intramedulary, intracardiac, intra-articular (joint), intrasynovial (joint fluid area), intracranial, intraspinal, and intrathecal (spinal fluids). Any known device useful for parenteral injection or infusion of drug formulations can be used to effect such administration.

Administration during in vivo treatment may be via any number of routes, including parenteral and oral, but preferably parenteral. Intracapsular, intravenous, intrathecal, and intraperitoneal routes of administration may be employed, generally intravenous is preferred. The skilled artisan will recognize that the route of administration may vary depending on the disorder to be treated.

Thus, the 4-1BBL and anti-4-1BB antibody may be administered in pharmaceutically acceptable formulations and in substantially non-toxic quantities. The present invention, therefore, also includes pharmaceutical compositions comprising a 4-1BBL and anti-4-1BB antibody of the present invention, including the 4-1BBL and anti-4-1BB antibody and biologically active fragments thereof, and a pharmaceutically acceptable carrier or diluent.

In another embodiment, administration of 4-1BBL and anti-4-1BB antibody can be to mucosal tissues by nasal application, by inhalation, ophthalmically, orally, rectally, vaginally, or by any other mode that results in the 4-1BBL and anti-4-1BB antibody contacting mucosal tissues.

Formulations for parenteral administration can be in the form of aqueous or non-aqueous isotonic sterile injection solutions, suspensions or fat emulsions. The parenteral form used for injection must be fluid to the extent that easy syringability exists. These solutions or suspensions can be prepared from sterile concentrated liquids, powders or granules.

Excipients useful in parenteral preparations also include, without limitation, stabilizing agents (e.g., carbohydrates, amino acids and polysorbates, such as 5% dextrose), solubilizing agents (e.g., cetrimide, sodium docusate, glyceryl monooleate, polyvinylpyrolidone (PVP) and polyethylene glycol (PEG)), surfactants (e.g., polysorbates, tocopherol PEG succinate, poloxamer and Cremophor™), buffers (e.g., acetates, citrates, phosphates, tartrates, lactates, succinates, amino acids and the like), antioxidants and preservatives (e.g., BHA, BHT, gentisic acids, vitamin E, ascorbic acid, sodium ascorbate and sulfur containing agents such as sulfites, bisulfites, metabisulfites, thioglycerols, thioglycolates and the like), tonicity agents (for adjusting physiological compatibility), suspending or viscosity agents, antibacterials (e.g., thimersol, benzethonium chloride, benzalkonium chloride, phenol, cresol and chlorobutanol), chelating agents, and administration aids (e.g., local anesthetics, anti-inflammatory agents, anti-clotting agents, vasoconstrictors for prolongation and agents that increase tissue permeability), and combinations thereof.

Injectable preparations include sterile aqueous solutions or dispersions and powders, which may be diluted or suspended in a sterile environment prior to use. Carriers such as solvents or dispersion media containing water, ethanol polyols, vegetable oils and the like may also be added to the compositions described herein. Coatings such as lecithins and surfactants may be used to maintain the proper fluidity of the composition. Isotonic agents such as sugars or sodium chloride may be added, as well as products intended to delay absorption of the active compounds, such as aluminum monostearate and gelatin. Sterile injectable solutions are prepared according to methods well known to those of skill in the art and can be filtered prior to storage and/or use. Sterile powders may be vacuum or freeze dried from a solution or suspension. Sustained-release preparations and formulations are also contemplated. Any material used in the compositions described herein should be pharmaceutically acceptable and substantially non-toxic in the amounts employed. Antimicrobial compounds may optionally be added to the preparations.

Parenteral formulations may also use hydrophobic carriers including, for example, fat emulsions and formulations containing lipids, lipospheres, vesicles, particles and liposomes. Fat emulsions include in addition to the above-mentioned excipients, a lipid and an aqueous phase, and additives such as emulsifiers (e.g., phospholipids, poloxamers, polysorbates, and polyoxyethylene castor oil), and osmotic agents (e.g., sodium chloride, glycerol, sorbitol, xylitol and glucose). Liposomes include natural or derived phospholipids and optionally stabilizing agents such as cholesterol.

Alternatively, the unit dosage of 4-1BBL and anti-4-1BB antibody of the present invention can be in a concentrated liquid, powder or granular form for ex tempore reconstitution in the appropriate pharmaceutically acceptable carrier at the time of delivery, and dilution where appropriate. In addition to the above-mentioned excipients, powder forms optionally include bulking agents (e.g., mannitol, glycine, lactose, sucrose, trehalose, dextran, hydroxyethyl starch, ficoll and gelatin), and cryo or lyoprotectants.

In intravenous (IV) use, a sterile formulation of 4-1BBL and anti-4-1BB antibody of the present invention and optionally one or more additives, including solubilizers or surfactants, can be dissolved or suspended in any of the commonly used intravenous fluids and administered by infusion. Intravenous fluids include, without limitation, physiological saline, phosphate buffered saline, 5% dextrose or Ringer's™ solution.

In intramuscular preparations, a sterile formulation of 4-1BBL and anti-4-1BB antibody of the present invention can be prepared and administered in a pharmaceutical diluent such as Water-for-Injection (WFI), physiological saline or 5% dextrose.

In one embodiment of the present invention, the 4-1BBL and anti-4-1BB antibody exist as an atomized dispersion for delivery by inhalation. The atomized dispersion of 4-1BBL and anti-4-1BB antibody typically contains carriers common for atomized or aerosolized dispersions, such as buffered saline and/or other compounds well known to those of skill in the art. The delivery of the 4-1BBL and anti-4-1BB antibody via inhalation has the effect of rapidly dispersing the 4-1BBL and anti-4-1BB antibody to a large area of mucosal tissues as well as quick absorption by the blood for circulation of the 4-1BBL and anti-4-1BB antibody. One example of a method of preparing an atomized dispersion is described in U.S. Pat. No. 6,187,344, entitled, “Powdered Pharmaceutical Formulations Having Improved Dispersibility,” which is hereby incorporated by reference in its entirety.

The 4-1BBL and anti-4-1BB antibody described herein can also be formulated in the form of a rectal or vaginal suppository. Typical carriers used in the formulation of the inactive portion of the suppository include polyethylene glycol, glycerine, cocoa butter, and/or other compounds well known to those of skill in the art. Although not wishing to be bound by theory, delivery of 4-1BBL and anti-4-1BB antibody via a suppository is hypothesized to have the effect of contacting a mucosal surface with the 4-1BBL and anti-4-1BB antibody for release to proximal mucosal tissues. Distal mucosal tissues also receive the 4-1BBL and anti-4-1BB antibody by diffusion. Other suppository formulations suitable for delivery of the 4-1BBL and anti-4-1BB antibody encompassed by the present invention are also contemplated.

Additionally, the 4-1BBL and anti-4-1BB antibody of the present invention may also be formulated in a liquid form. The liquid can be for oral dosage, for ophthalmic or nasal dosage as drops, or for use as an enema or douche. When the 4-1BBL and anti-4-1BB antibody are formulated as a liquid, the liquid can be either a solution or a suspension of the 4-1BBL and anti-4-1BB antibody. There is a variety of suitable formulations for the solution or suspension of the 4-1BBL and anti-4-1BB antibody that are well know to those of skill in the art, depending on the intended use thereof. Liquid formulations for oral administration prepared in water or other aqueous vehicles may contain various suspending agents such as methylcellulose, alginates, tragacanth, pectin, kelgin, carrageenan, acacia, polyvinylpyrrolidone, and polyvinyl alcohol. The liquid formulations may also include solutions, emulsions, syrups and elixirs containing, together with the active compound(s), wetting agents, sweeteners, and coloring and flavoring agents. Various liquid and powder formulations can be prepared by conventional methods for inhalation into the lungs of the mammal to be treated.

Delivery of the described 4-1BBL and anti-4-1BB antibody in liquid form via oral dosage exposes the mucosa of the gastrointestinal and urogenital tracts to the 4-1BBL and anti-4-1BB antibody. A suitable dose, stabilized to resist the pH extremes of the stomach, delivers the 4-1BBL and anti-4-1BB antibody to all parts of the gastrointestinal tract, especially the intestines. Any method of stabilizing the 4-1BBL and anti-4-1BB antibody in a liquid oral dosage such that the effective delivery of the composition is distributed along the gastrointestinal tract is contemplated for use with the 4-1BBL and anti-4-1BB antibody described herein.

Delivery of the described 4-1BBL and anti-4-1BB antibody in liquid form via ophthalmic drops exposes the mucosa of the eyes and associated tissues to the 4-1BBL and anti-4-1BB antibody. A typical liquid carrier for eye drops is buffered and contains other compounds well known and easily identifiable to those of skill in the art.

Delivery of the described 4-1BBL and anti-4-1BB antibody in liquid form via nasal drops exposes the mucosa of the nose and sinuses and associated tissues to the 4-1BBL and anti-4-1BB antibody. Liquid carriers for nasal drops are typically various forms of buffered saline.

### Administration

Administration of the formulations discussed above can be practiced in vitro or in vivo. When practiced in vitro, any sterile, non-toxic route of administration may be used. When practiced in vivo, systemic administration of the formulations discussed above may be achieved advantageously by subcutaneous, intravenous, intramuscular, intraocular, oral, transmucosal, or transdermal routes, such as, for example, by injection or by means of a controlled release mechanism. Examples of controlled release mechanisms include polymers, gels, microspheres, liposomes, tablets, capsules, suppositories, pumps, syringes, ocular inserts, transdermal formulations, lotions, creams, transnasal sprays, hydrophilic gums, microcapsules, inhalants, and colloidal drug delivery systems.

While the 4-1BBL and anti-4-1BB antibody of the present invention may be administered systemically in the manners discussed above, in equally preferred embodiments of each of the methods set forth herein the 4-1BBL and anti-4-1BB antibody may be administered in a targeted fashion to a particular location in the subject, such as directly to the interior of the intestine or to the tissues of the intestine, to visceral adipose tissue or to subcutaneous adipose tissue, or directly into the blood stream, into or near a tumor or cancer, into or near a lymph node or into the bone marrow.

The therapeutically effective amount of the 4-1BBL and anti-4-1BB antibody of the present invention varies depending upon the physical characteristics of the patient, the severity of the patient's symptoms, the disease or condition to be treated or inhibited, and the formulation and the means used to administer the polypeptides. The specific dose for a given subject is usually set by the judgment of the attending physician. However, a therapeutically effective amount of the polypeptides of the present invention is typically between about 0.5 mg/kg body weight to 500 mg/kg body weight, preferably from 1 to 100 mg/kg, more preferably from 3 to 50 mg/kg, 3 to 30 mg/kg or 3 to 15 mg/kg, regardless of the formulation. In equally preferred embodiments, a therapeutically effective amount is about 0.5, 1, 3, 5, 10, 15, 20, 25, 30, 35, 40, 45 or 50 mg/kg body weight, regardless of the formulation. In some situations, a dose less than 0.5 mg/kg body weight may be effective.

Suitable frequencies for administering a 4-1BBL and anti-4-1BB antibody of the invention to a subject may also vary based on the severity of the patient's symptoms, the disease or condition to be treated or inhibited, and the formulation and the means used to administer the polypeptide or vector. However, administration frequencies include 4, 3, 2 or once daily, every other day, every third day, every fourth day, every fifth day, every sixth day, once weekly, every eight days, every nine days, every ten days, bi-weekly, monthly and bi-monthly, yearly, and less frequent doses including a single dose.

The doses may be administered at the normal rate selected for a particular means of administration, or the doses may be administered at a slower rate, such as over a period of minutes, hours or days. Particular periods of administration include 5, 10, 15, 20, 25, 50, 40, 50 or 60 minutes, and 1, 2, 3, 4, 5, 6, 7, 8, 9, 10 or more hours.

Throughout this application, the term “about” is used to indicate that a value includes the standard deviation of error for the device or method being employed to determine the value. The term “about,” as used herein, should generally be understood to refer to both numbers in a range of numerals. Moreover, all numerical ranges herein should be understood to include each whole integer within the range.

The terms “a” and “an,” when used in conjunction with the word “comprising” in the claims or specification, denotes one or more, unless specifically noted.

It is specifically contemplated that any embodiments described in the Examples section are included as an embodiment of the invention.

The following examples illustrate how external signals may regulate 4-1BB expression on human B cells and possibly define the biological effect of 4-1BB-mediated co-stimulation on human B cells. The first-tier regulation of 4-1BB expression on human B cells may be initiated by B cell receptor (BCR) stimulation while CD40 ligation and cytokines provide the second tier regulation. The presence of 4-1BB on human B cells may be functionally relevant since stimulation with human 4-1BBL transfected cell lines at the time of activation, induces proliferation and protects these cells from activation- and chemotherapy-induced apoptosis. Accordingly, results obtained from these examples may demonstrate that 4-1BB co-stimulation plays a direct role in defining the fate of antigen-stimulated human B cells.

### EXAMPLES

### Example 1

### 1. Cell Preparation

Buffy coats from healthy donors were purchased (Biological Specialty Corporation, Colmar, Pa.) and peripheral blood mononuclear cells (PBMC) were prepared by density centrifugation (Ficoll-Paque, Amersham). B lymphocytes were purified from PBMC by negative selection using B cell isolation kit II (Miltenyi Biotec, Auburn, Calif.) and T lymphocytes were purified by positive selection using CD3 microbeads (Miltenyi Biotec) according to the manufacturer's instructions. Purity of cell separations were typically >98% for B and T lymphocytes with less than 0.2% contamination of CD3⁺ T cells in purified B cell populations (as assessed by flow cytometry). For the isolation of naïve and memory B cell subsets, CD19⁺ cells were positively selected using a CD19 multisort kit (Miltenyi Biotec), followed by separation of CD19⁺CD27⁺ and CD19⁺CD27⁻ cell subsets using CD27 microbeads (Miltenyi Biotec) according to the manufacturer's instructions.

### 2. B Cell Activation

All in vitro cell cultures were performed in RPMI 1640 supplemented with 10% fetal calf serum (FCS), 1% P/S, 1% HEPES and 1% Glutamax.

Stimulation of PBMC: 2×10⁶ PBMC were stimulated with 2 μg/ml of pokeweed mitogen (PWM) (Sigma), 5 μg/ml of phytohemagglutinin (PHA) (Calbiochem), 25 ng/ml of Phorbol myristate acetate (PMA)/1 μg/ml of Ionomycin (Sigma), 2.5 μg/ml of CpG (InvivoGen) or 5 μg/ml of lipopolysaccharide (LPS) (Sigma) in 24-well plates. After 72 hours, PBMC were harvested, washed and assessed for 4-1BB expression by flow cytometry.

B/T cell co-culture experiments: Purified B cells (1×10⁶) and T cells (2×10⁶) were co-cultured and stimulated with PWM under the conditions described above. Direct B/T cell interactions were inhibited by the addition of transwell membranes. In brief, 2×10⁶T cells were added to the upper chamber of transwell plates (polyester membranes 6.5-mm, 0.4-μm, Corning Costar) and 1×10⁶ B cells were added to the lower chamber. To block CD40-CD40 ligand (CD40L) interactions, various concentrations (1-20 μg/ml) of purified anti-CD40L (BD Biosciences) monoclonal antibody (mAb) were added to PBMC and B/T cell co-cultures.

BCR-mediated stimulation experiments: Purified B cells or B cell subsets were cultured at a concentration of 1×10⁶/ml in 48-well or 96-well flat bottom plates. B cells were activated with 10 μg/ml anti-Igs F(ab′)₂ fragments (goat anti-human IgA+IgG+IgM (H+L), Jackson ImmunoResearch Laboratories), with or without the addition of 1 μg/ml purified goat anti-human CD40 antibody (R&D Systems). The following human recombinant cytokines were used to evaluate their impact on B cell stimulation: 100 u/ml of interleukin-2 (IL-2) (Proleukine, Chiron Corporation, Emeryville, Calif.), 20 ng/ml of IL-4 (R&D systems), 1 ng/ml of IL-6 (BD Bioscience), 50 ng/ml of IL-10 (eBioscience), 50 ng/ml of IL-15 (R&D systems), 100 ng/ml of IL-21 (Biosource International, Inc. Camarillo, Calif.), 500 u/ml of IFN-γ (eBioscience) and 50 ng/ml of TNF-α (BD Bioscience).

### 3. Flow Cytometry

B cells were phenotyped by staining with directly conjugated mouse anti-human mAbs against CD3, CD19, CD32, CD69, CD86, CD95, 4-1BB, 4-1BBL (all BD Bioscience), CD25, CD71, and CD27 (all eBioscience) mAb. Directly conjugated mouse IgGs were used as isotype controls. Labeled cells were acquired on a LSRII flow cytometer and analyzed with FACS Diva (BD Biosciences) and Winlist (Verity Software House) Software.

To evaluate cell proliferation by flow cytometry, B cells and separate B cell subsets were labeled with CFSE (Alexis Biochemicals) according to the manufacturer's instructions. CFSE labeled cells were cultured as described above and indicated in appropriate figure legends. At day 3 or day 4 of culture, cells were harvested, stained with indicated cell surface markers and analyzed by flow cytometry. Cell cycle analysis was performed using a bromodeoxyuridine (BrdU) flow kit (BD Biosciences). In brief, activated purified B cells were pulsed with 10 μM BrdU. After 4 hours, cells were harvested and cell surface stained with mAbs indicated in figure legends, followed by intracellular staining with anti-BrdU mAb. 7-AAD was used to evaluate DNA content. B cell apoptosis was determined by staining with Annexin V/7-AAD (Annexin V-PE apoptosis detection kit I, BD Biosciences) according to the manufacturer's recommendations. Accucount particles (Spherotech) were added before analyzing samples to obtain accurate absolute cell numbers which were calculated by the manufacturer's instructions.

### 4. B Cell Function

Generation of 4-1BBL transfectants: Human 4-1BBL cDNA was obtained by RT-PCR from total RNA extracted from human PBMC and sub-cloned into a mammalian expression vector (pCDNA3.1, Invitrogen, Huntsville, Ala.). P815 cells were transfected with human 4-1BBL using Lipofectamine (Invitrogen). After selection with G418 (800 μg/ml) for 1-2 weeks, drug-resistant cells were FACS sorted for 4-1BBL expression. 4-1BBL positive cells were further cloned by limiting dilution. A clone with high levels of 4-1BBL expression (hereafter called P815-4-1BBL) was selected and used in subsequent functional B cell experiments. P815 cells transfected with vector alone were used as a negative control (hereafter called P815-mock).

4-1BB-4-1BBL interaction experiments: Gamma-irradiated (100 gy) P815-4-1BBL cells or P815-mock cells were cultured with purified human B cells for 3-7 days. All cytokines and stimuli were added at the initiation of culture and are indicated in figure legends.

Tritiated thymidine (³H-TdR) incorporation assays: Purified B cells were seeded at 2×10⁵/well in triplicate wells in a 96 well flat-bottom plate and stimulated as described under “B Cell Activation” above in the presence of irradiated P815-mock or P815-4-1BBL cells. To block the interaction of 4-1BB with 4-1BB ligand, 10 μg/ml of soluble 4-1BB protein (Prospec, Rehovot Israel) was added at the initiation of culture. ³H-TdR (37 Kbq/well) was added 16 hours before completion of the experiment and thymidine incorporation was measured using a liquid scintillation counter (Wallac).

Induction of apoptosis: Activation-induced cell apoptosis was performed by culturing B cells with anti-Ig alone, or in combination with IFN-γ or anti-CD40 as described under “B cell activation experiments”. For chemotherapy-induced apoptosis, B cells were activated with anti-Ig/anti-CD40 for 3 days and subsequently incubated with various concentration of Doxorubicin (Bedford Laboratories) for 24 hours. In growth factor deprivation experiments, culture supernatants of anti-Ig/anti-CD40 activated B cells were replaced with fresh medium and cells were further cultured for 24 hours.

### 5. Western Blot Analysis

Purified B cells were stimulated with anti-Ig, anti-Ig/IFN-γ, or anti-Ig/anti-CD40 in the presence of irradiated P815-4-1BBL transfectants for 3 days. Next, CD19⁺ cells were positively selected using CD19 microbeads. Proteins were extracted with lysis buffer (50 mM Tris PH 8.0, 150 mM NaCl, 1% NP-40) and protease inhibitors cocktail (Roche Applied Science) was added just prior to use. Equal amounts of protein from each sample were loaded on 4%-20% SDS-PAGE gels and subsequently separated and transferred to nitrocellulose membranes (Amersham). Membranes were blocked and incubated overnight at 4° C. with murine mAb against BCL-2 and rabbit polyclonal antibody against MCL-1 and BCL-XL (Santa Cruz Biotechnology). Murine mAb against β-actin was used as an internal control. Membranes were then washed, incubated with goat anti-mouse or rabbit-horseradish peroxidase (HRP) antibody and developed by chemiluminescence using a Supersignal West Femto kit (Pierce).

### 6. Reverse Transcriptase-Polymerase Chain Reaction (RT-PCR)

Total RNA was extracted from anti-Ig/anti-CD40 activated B cells at indicated time points using RNeasy mini kit (Qiagen), reverse transcribed with AMV reverse transcriptase (Roche Applied Science) and amplified with specific primers to β-actin (Forward primer: 5′-CATGTACGTTGCTATCCAGGC-3′ (SEQ ID NO:1), Reverse primer: 5′-CTCCTTAATGTCACGCACGAT-3′ (SEQ ID NO:2)) and human 4-1BB (Forward primer: 5′-ACCTGTGCCAGATTTCAT-3′ (SEQ ID NO:3), Reverse primer: 5′-CAGCCCTATTGACTTCCA-3′ (SEQ ID NO:4)).

### 7. Statistical Analysis

Repeated measures model was used to compare 4-1BB expression under different conditions (reagents) so that the within sample (donor) correlation is accounted for. The maximum likelihood based estimation and unstructured covariance matrix option for the mixed model as implemented in SAS PROC MIXED were used in the computation.

Residual diagnosis such as Q-Q plot was used to check model fit. The model encompasses t-test and paired t-test when there were no missing values.

### Example 2

### 4-1BB Expression on Human B Cells Following Antigen Exposure and Expression is Regulated by Cognate Interactions Between CD40-CD40L and Pro-Inflammatory Cytokines

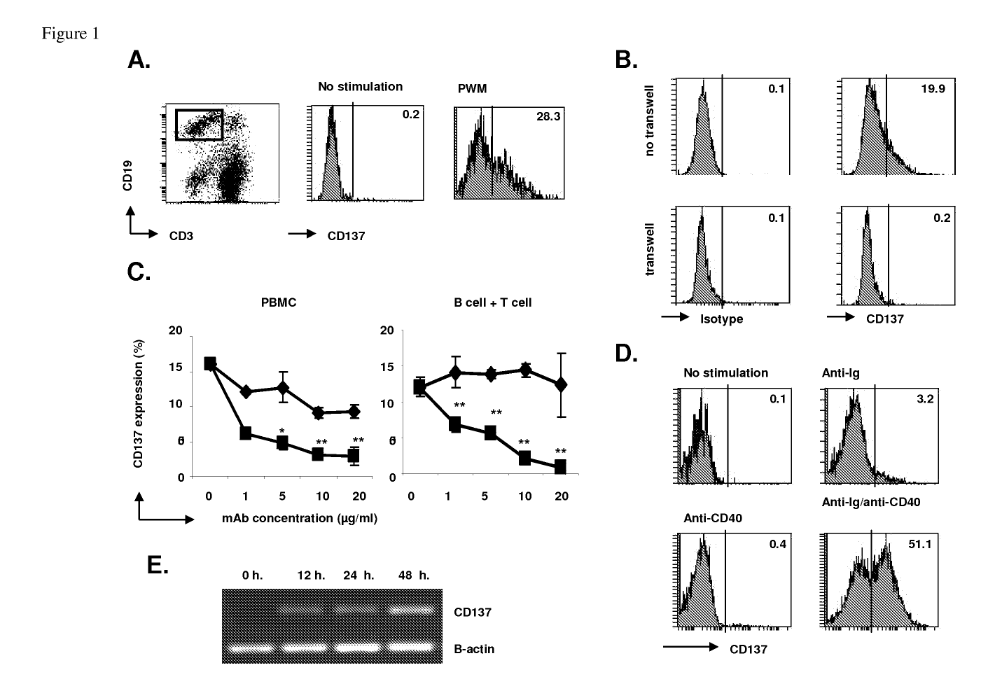

To initially characterize activating signals required to induce expression of 4-1BB on human B cells, whole PBMC were stimulated with various mitogenic stimuli. Human B cells were found to up-regulate 4-1BB in the presence of PWM (FIG. 1A) while stimulation with PMA, PHA, LPS, PMA/Ionomycin or CpG were not effective (data not shown). Histograms in FIG. 1A indicate percentage 4-1BB expression on gated B (CD19⁺) cells in whole PBMC. Data are representative of at least 5 individual experiments.

Because PWM is recognized to activate both T cells and B cells, we next sought to determine if 4-1BB expression on human B cells is T cell-dependent. We observed that purified B cells did not up-regulate 4-1BB in the presence of PWM (data not shown). However, PWM stimulation of cultures containing isolated T cells and B cells induced B cell associated 4-1BB. This expression of 4-1BB is cell-to-cell contact dependent since B cells separated from T cells, by a transwell membrane, did not up-regulate 4-1BB (FIG. 1B). Histograms in FIG. 1B indicate percentage 4-1BB expression on gated B (CD19⁺) cells. Data are representative of 4 individual experiments. As the CD40-CD40L co-stimulatory pathway is integral to T cell-dependent B cell function, we defined the relative import of CD40-CD40L interactions in the up-regulation of 4-1BB on B cells. The addition of escalating concentrations of CD40L mAb to both PWM stimulated PBMC and purified T/B cell co-cultures, significantly reduced the percentage of 4-1BB expression (P<0.0001) in a dose-dependent fashion (FIG. 1C). Data in FIG. 1C are presented as mean±SD and are representative of 5 individual experiments. To confirm the significance of CD40-CD40L interactions in the induction of 4-1BB, purified B cells were stimulated with anti-Ig in the presence or absence of anti-CD40 stimulating antibody. Resting (non-stimulated) B cells and B cells stimulated with anti-CD40 antibody alone did not up-regulate 4-1BB. However, B cells stimulated with anti-Ig up-regulated low levels of 4-1BB while 4-1BB expression levels were dramatically enhanced with concurrent CD40 ligation (P=0.0003) at both the protein and mRNA level (FIG. 1D-E). 4-1BB is induced on human B cells upon BCR stimulation with enhancement of cell surface expression levels following CD40 ligation with agonistic anti-CD40 antibody (FIG. 1D). Histograms in FIG. 1D indicate percentage 4-1BB expression on gated B (CD19⁺) cells. RT-PCR confirmed the up-regulation of 4-1BB mRNA in anti-Ig/anti-CD40 stimulated B cells after 12 hours of culture (FIG. 1E). Data in FIG. 1E are representative of 3 individual experiments. *P<0.05 and “P<0.0001. These data demonstrate that 4-1BB is induced on human B cells following antigen exposure and that expression levels are enhanced by cognate interactions between CD40 and CD40L. Importantly, 4-1BB expression levels on B cells varied among different healthy individuals, ranging from 3.2% to 30.7% (N=10; median=13.9%) and from 10.7% to 62.2% (N=14; median=43.1%) upon stimulation with anti-Ig or anti-Ig/anti-CD40 respectively.

Second Level of Regulatory Control of B Cell-Associated 4-1BB Expression by Cytokines

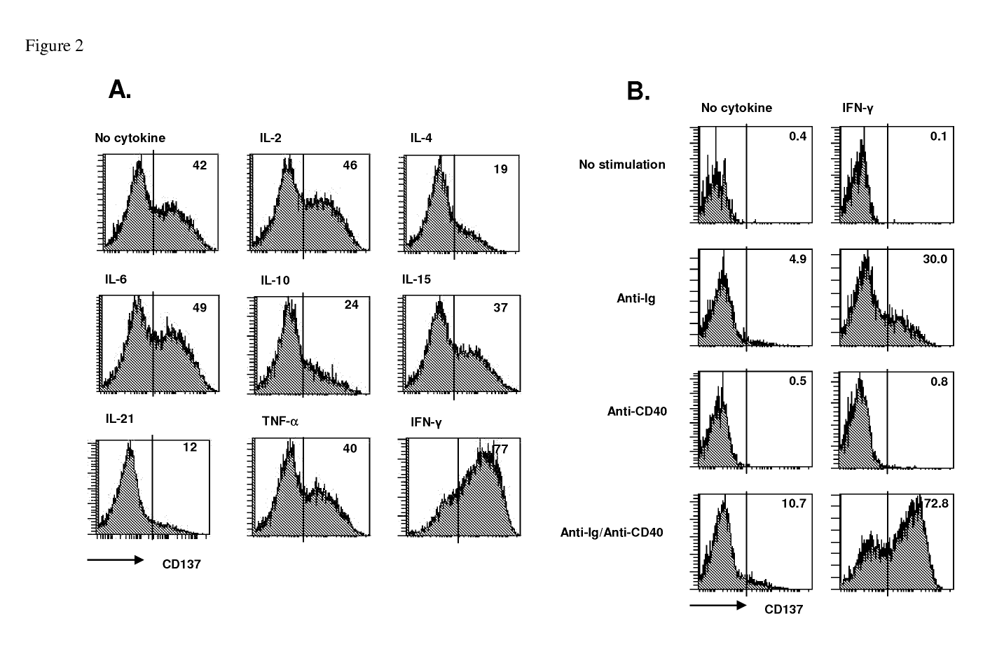

Because cytokines are recognized to influence human B cell function, their impact on 4-1BB expression was evaluated. Purified B cells were stimulated with anti-Ig/anti-CD40 antibodies in combination with defined cytokines recognized to mediate B cell function. IL-2, TNF-α, IL-6 and IL-15 did not directly affect 4-1BB expression on human B cells. However, co-culture of anti-Ig/anti-CD40 stimulated B cells with IFN-γ dramatically enhanced (P<0.0001) the percentage of 4-1BB expressing cells while IL-4 (P<0.0001), IL-10 (P=0.0188) and IL-21 induced the opposite effect (FIG. 2A). Similar to the effects of CD40 ligation, the ability of IFN-γ to enhance 4-1BB expression is dependent upon BCR signaling (FIG. 2B) since B cells in the presence of IFN-γ alone did not up-regulate 4-1BB. These data suggest that exposure to antigen is required for 4-1BB expression and that CD40 signaling and select cytokines provide a second level of regulatory control for 4-1BB expression on human B cells.

### Example 3

### 4-1BB is Preferentially Expressed on Activated B Cells of Naïve Origin

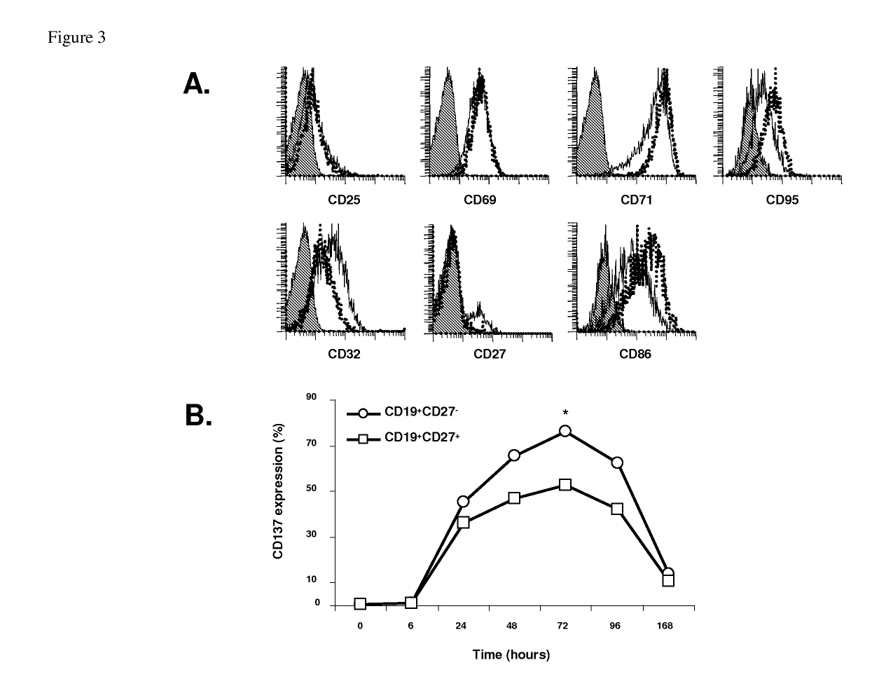

As a first step in characterizing the function of 4-1BB on human B cells, the cell surface phenotype of 4-1BB⁺ and 4-1BB⁻ B cells were compared. Anti-Ig/anti-CD40 stimulated 4-1BB⁺ B cells demonstrated elevated levels of CD71, CD86, and CD95 but diminished expression of CD32 (FIG. 3A). Interestingly, while 4-1BB⁻ B cells expressed small amounts of CD27, this marker was virtually absent on 4-1BB⁺ B cells. Since CD27 distinguishes naïve B cells (CD19⁺CD27⁻) from memory B cells (CD19⁺CD27⁺), we evaluated if 4-1BB was differentially up-regulated on cells originating from these distinct populations. Naïve and memory B cells were purified based on their levels of CD27 expression, cultured in the presence of anti-Ig/anti-CD40, and harvested at defined time intervals. In both cell populations, 4-1BB expression was present on day one and maintained at semi-consistent levels for four days. By day seven, the expression of 4-1BB returned to baseline. Overall, the percentage of 4-1BB⁺ B cells was higher in cultures originating from CD27⁻ versus CD27⁺ B cells at every time point tested (FIG. 3B) with P=0.0014 on day four. These data demonstrate that following anti-Ig/CD40 ligation, 4-1BB is preferentially, but not exclusively, found on activated B cells originating from the naïeve B cell subset.

### Example 4

### 4-1BB Expression is Associated with Enhanced B Cell Activation

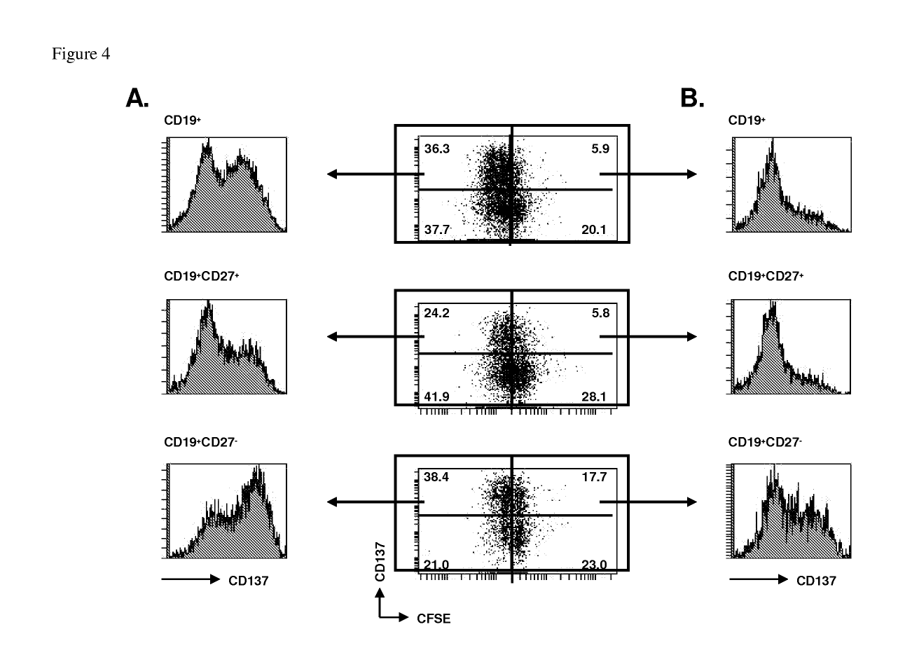

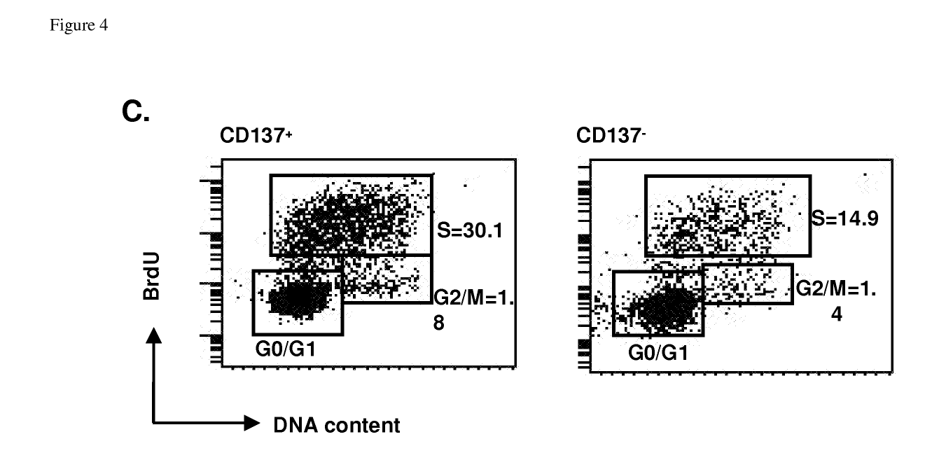

It is reported that the differentiation of human B cells into effector immunoglobulin secreting cells (ISCs) requires cell division (26-27). Since 4-1BB expression correlated with enhanced levels of CD71, a marker associated with cell proliferation, we sought to determine if 4-1BB expression on human B cells is associated with B cell division. Based on CFSE dilution, higher levels of 4-1BB are expressed on divided (FIG. 4A) compared to non-divided B cells (FIG. 4B) were observed. Specifically, 4-1BB is predominantly expressed on divided memory B cells. In contrast, both divided and non-divided naïve B cells express high levels of 4-1BB although higher 4-1BB expression levels are found on naïve B cells with low CFSE intensity. Importantly, the expression of 4-1BB does not solely rely on B cell division since 4-1BB is present on the B cell surface prior to cell division and blocking of B cell division with mitomycin C does not abrogate the ability of anti-Ig/anti-CD40 activated B cells to up-regulate 4-1BB (data not shown). Furthermore, cell cycle analysis confirmed that more 4-1BB⁺ B cells (30%) are present in the S phase than 4-1BB⁻ B cells (15%; FIG. 4C). These data demonstrate that following anti-Ig/anti-CD40 activation, 4-1BB is preferentially, but not exclusively, found on highly activated B cells originating from the naïve B cell subset.

### Example 5

### 4-1BB Ligation Enhances B Cell Proliferation

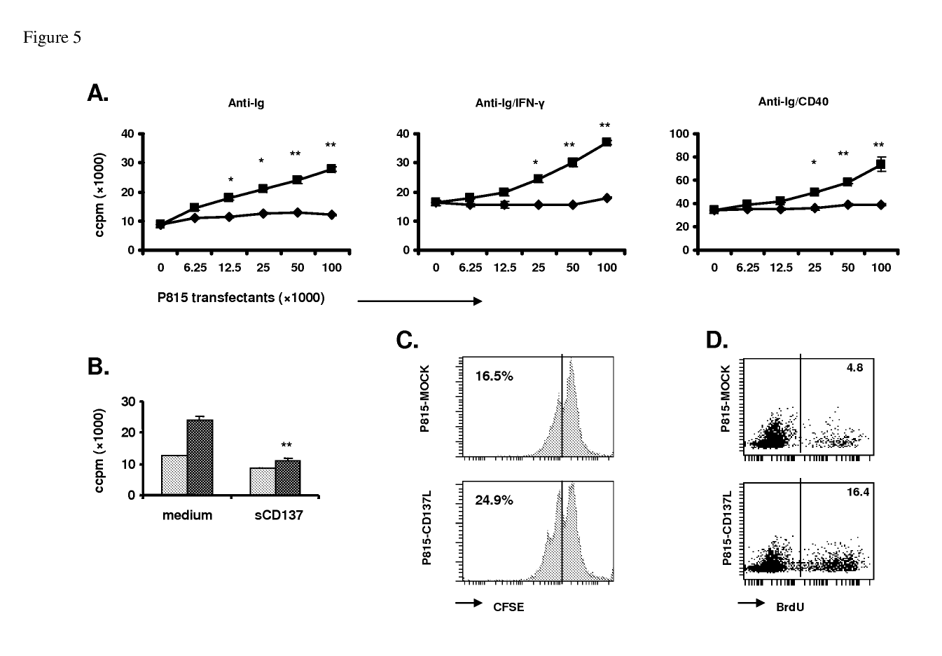

To expand upon the observation that 4-1BB expression on B cells is associated with B cell activation and cell division, the induction of proliferation by 4-1BB-mediated co-stimulation of activated B cells was evaluated. In order to study the effect of 4-1BB-mediated co-stimulation on human B cells, P815 cell clones-expressing human 4-1BBL were generated. These cells were recognized by both mouse anti-human 4-1BBL mAb and human 4-1BB fusion protein, confirming cell surface expression of 4-1BBL and the ability to interact with human 4-1BB receptor respectively (data not shown). Purified B cells were stimulated with anti-Ig, anti-Ig/IFN-γ or anti-Ig/anti-CD40 in the presence of P815-4-1BBL or P815-mock cells. In comparison to B cell cultures with P815-mock, the presence of P815-4-1BBL significantly enhanced ³H-TdR incorporation in all three culture conditions (FIG. 5A). The specificity of the observed effects was confirmed in 4-1BB-4-1BBL blocking experiments, where the addition of soluble human 4-1BB protein to anti-Ig stimulated B cell cultures completely abrogated the observed differences (FIG. 5B). These data suggest that the observed changes in thymidine incorporation are specifically mediated through 4-1BB-4-1BBL interactions.

Subsequent studies using CFSE dilution and cell cycle analysis confirmed that 4-1BB mediates B cell proliferation. Specifically, anti-Ig activated B cells only completed one cell division in the presence of P815-mock whereas in the presence of 4-1BB co-stimulation (P815-4-1BBL), cells completed two or more divisions and exhibited a greater percent of B cells in the S phase of the cell cycle compared to those cultured with P815-mock (FIGS. 5C and 5D). Despite clear differences in thymidine incorporation, no differences were observed in B cell division and cell cycle status when anti-Ig/anti-CD40 activated human B cells were cultured in the presence of P815-4-1BBL (data not shown) which suggests that the initial B cell activation signal determines the net effect of 4-1BB-mediated B cell proliferation.

### Example 6

### 4-1BB Ligation Can Promote Human B Cell Survival and is Associated with the Up-Regulation of Anti-Apoptotic Proteins

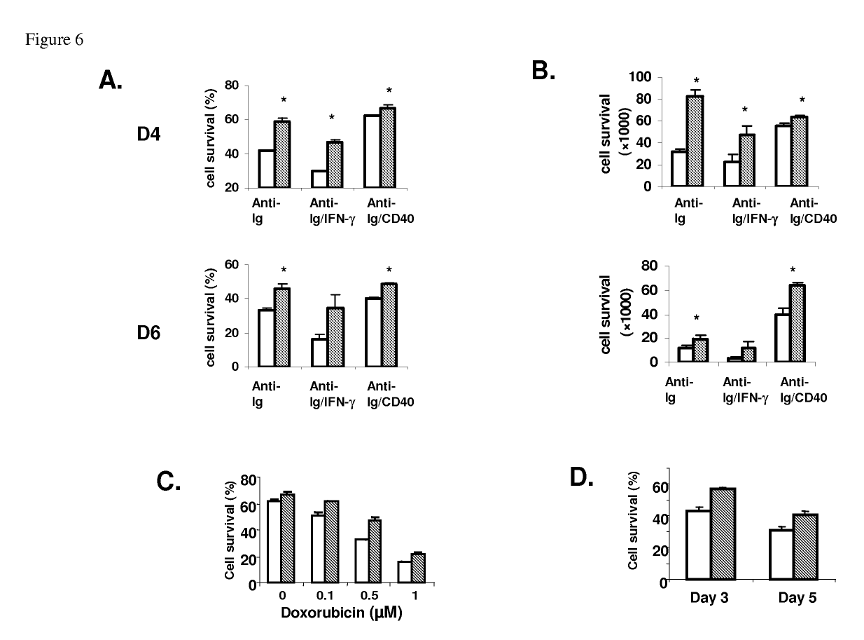

Based on the observation that 4-1BB ligation in B cell cultures exposed to anti-Ig/anti-CD40 enhanced thymidine incorporation, yet failed to induce clear cell division and cell cycle progression, we postulated that 4-1BB may provide a survival advantage for B cells. In order to test this hypothesis, purified B cells were stimulated with anti-Ig, anti-Ig/IFN-γ, or anti-Ig/anti-CD40 in the presence of mock or 4-1BBL transfected P815 cells. Cell survival was assessed at various time points by flow cytometric staining with Annexin V and 7-AAD. In the presence of P815-4-1BBL, the percentage of surviving B cells (as determined by Annexin V77-AAD⁻) improved in all three culture conditions (FIG. 6A) at both day four and day six.

Since different B cell activation stimuli differentially affect 4-1BB-mediated B cell proliferation, the absolute B cell number was also determined. Similar to the percentage of cell survival, the absolute number of live B cells was greatly enhanced in the presence of P815-4-1BBL with the most dramatic changes at day four for B cells stimulated with anti-Ig and anti-Ig/IFN-γ and at day six for B cells stimulated with anti-Ig/anti-CD40 (FIG. 6B). Furthermore, 4-1BBL stimulated B cells maintain their survival advantage in the presence of chemotherapeutic Doxorubicin or growth factor deprivation (FIG. 6C-D). These data demonstrate that ligation of B cell associated 4-1BB inhibits human B cell death induced by mechanically distinct signals.

### Example 7

### Association of 4-1BB Ligation with the Up-Regulation of Anti-Apoptotic Proteins

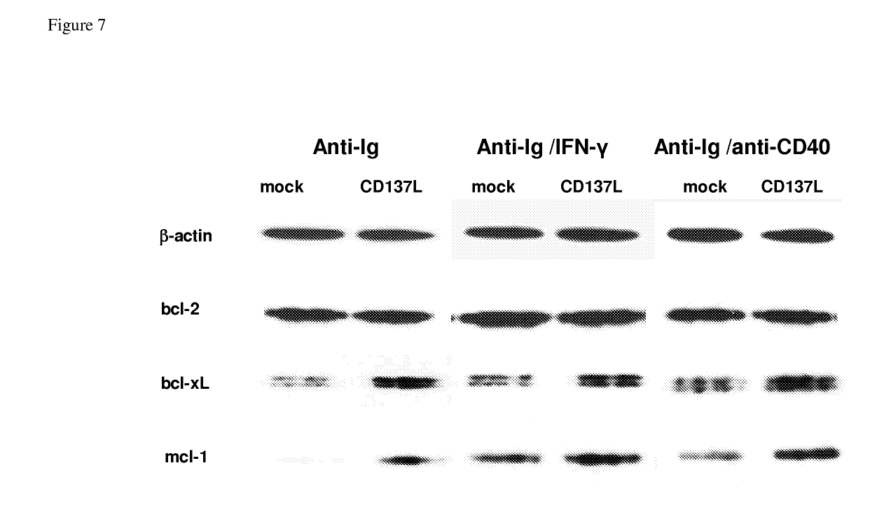

To begin to unravel the potential molecular mechanisms which may be involved in 4-1BB-mediated B cell survival, the expression of various anti- and pro-apoptotic Bcl-2 family-derived proteins was analyzed. No consistent changes were observed in pro-apoptotic proteins (e.g., Bax, Bad, Bik and Bim) after 4-1BB ligation (data not shown). In contrast, the expression of anti-apoptotic proteins Bcl-x1 and Mcl-1 was up-regulated during 4-1BB ligation while no difference in expression was observed for Bcl-2 (FIG. 7). Interestingly, no differences in bcl-x1 and mcl-1 mRNA expression levels were detected by quantitative PCR which suggests that 4-1BB-mediated differences in anti-apoptotic protein expression are more likely to be regulated at a post-transcriptional level (data not shown).

### Example 8

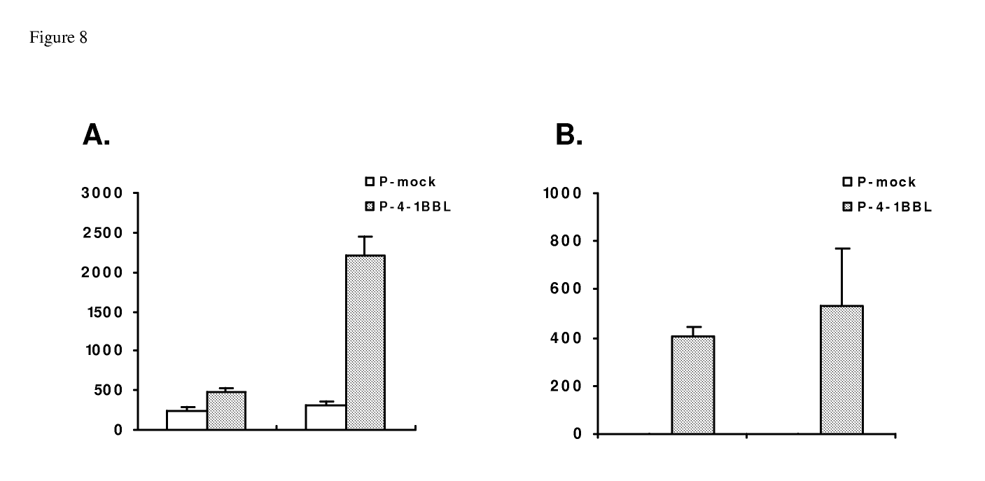

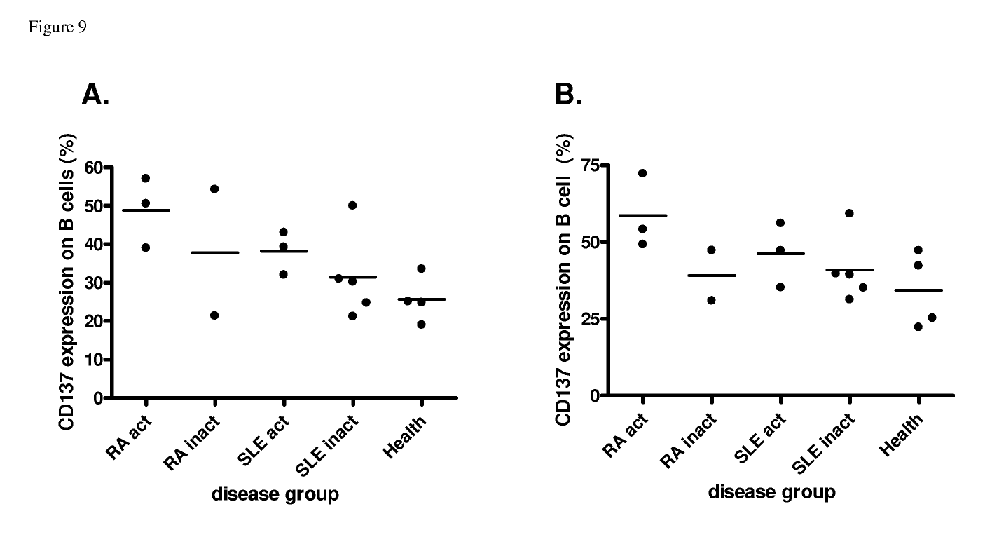

It has been previously shown that human B lymphocytes upregulate 4-1BB upon anti-BCR stimulation. Co-stimulation of 4-1BB on anti-Ig activated B cells induces cell proliferation, promotes cell survival and cytokine production. Recently, it was found that 4-1BB significantly enhances the production of total IgM and IgG by anti-Ig activated B cells from healthy donors. (FIG. 8). Furthermore, we determined anti-BCR activation-induced 4-1BB expression on B cells from patients with autoimmune diseases, e.g., RA and SLE. Our data suggests that the patients with active RA disease upregulate B cell-associated 4-1BB (CD137) expression at a level significantly higher than in a healthy donor (FIG. 9).

As demonstrated hereinabove, despite the reported absence of 4-1BB on murine B cells, 4-1BB is expressed on human B cells. Expression requires initial signaling through the BCR and is enhanced by both CD40-CD40L interactions and IFN-γ and inhibited by IL-4 and IL-10. Generally, 4-1BB-expressing B cells are of naïve origin and have an activated phenotype, defined by enhanced expression of CD71, CD86 and CD95. Ligation of 4-1BB on the surface of human B cells induces B cell proliferation and protects against activation-induced cell death. These data demonstrate that, following antigen stimulation, 4-1BB may contribute to downstream B cell function and may be functionally relevant in the regulation of human B cell proliferation and survival.

These studies also reveal that 4-1BB is expressed on activated B cells following BCR stimulation. Cognate help from T cells through CD40-CD40L interaction and/or cytokines are important for regulation of 4-1BB expression on human B cells. Among the cytokines tested, only the Th1 cytokine IFN-γ enhanced 4-1BB expression, while IL-4 and IL-10 inhibited 4-1BB expression. Importantly, anti-CD40 stimulation and IFN-γ enhanced the level of 4-1BB expression in an additive, but not synergistic fashion and neither anti-CD40 stimulation nor cytokine alone were capable of inducing B cell expression of 4-1BB in the absence of BCR stimulation. In addition, polyclonal stimulation of human B cells with CpG, which stimulates TLR9-mediated B cell proliferation and differentiation in the absence of antigen, fails to up-regulate 4-1BB (data not shown). These data suggest that (i) 4-1BB expression on human B cell is tightly controlled; (ii) strictly dependent upon antigen encounter; and that (iii) the BCR serves as the initial “switch” which enables up-regulation of 4-1BB on the B cell surface.

Similar to T cell-associated 4-1BB, B cells transiently up-regulate 4-1BB with detectable cell surface levels after 24 hours of activation, maximal expression levels by day three or four and a return to baseline levels by day seven. This study demonstrates that 4-1BB⁺ B cells are phenotypically associated with enhanced expression of CD71, CD86 and CD95, while CD32 expression is decreased, which implies that 4-1BB⁺ B cells are highly activated. Furthermore, divided B cells express higher levels of 4-1BB compared to non-divided B cells, although 4-1BB expression is not dependent on B cell division. Interestingly, upon anti-Ig/anti CD40 stimulation, naïve B cells are more prone to enhanced 4-1BB expression than memory B cells. We postulate that 4-1BB-mediated co-stimulation may be especially important for naïve B cell regulation since naïve and memory B cells require different signals for cellular activation and differentiation (28, 29).

The functional effects of 4-1BB-mediated co-stimulation on T cell proliferation and survival are well documented (30-32) and here we demonstrate that 4-1BB on human B cells mediates analogous functional changes. ³H-thymidine incorporation by anti-Ig, anti-Ig/IFN-γ or anti-Ig/anti-CD40 stimulated B cells is significantly enhanced upon ligation with 4-1BB. Interestingly, B cell division and cell cycle progression are differently affected depending on the initial B cell activation signal. For example, B cell division is enhanced through 4-1BB ligation on B cells which have been stimulated with anti-Ig alone. In contrast, B cell division and cell cycle are not affected by 4-1BB ligation among B cells which are stimulated with both anti-Ig and anti-CD40, despite significantly higher 4-1BB expression levels. Since CD40 signaling is known to mediate B cell proliferation and survival (33), delicate 4-1BB-4-1BBL mediated enhancements in cell division and cell cycle progression may be masked by the potent proliferative effects of the CD40 signal itself. Despite the fact that various B cell stimuli, e.g. anti-Ig, anti-Ig/IFN-γ, anti-Ig/anti-CD40, differently impact on 4-1BB-mediated B cell proliferation, B cell survival was improved by 4-1BB ligation in all culture conditions. Specifically, greater improvement in B cell survival was observed among B cells activated by anti-Ig alone or in combination with IFN-γ, compared to those activated by anti-Ig/anti-CD40. Overall, 4-1BB-mediated changes in B cell function depend on initial B cell activation and are the net effect of both 4-1BB-mediated B cell proliferation and B cell survival.

According to the two phase model of B cell activation proposed by Baumgarth N., antigen activation of B cells leads to clonal expansion independent of T cell help during the early phase of an antigen-specific immune response (34). Antigen presenting cells (APCs) and factors produced by APCs affect this early activation response. For example, IL-12, GM-CSF and IFN-γ produced by APCs are important for the induction of T-cell-independent B cell responses (35, 36). Additionally, the B cell-activating factor belonging to the TNF family (BAFF) induces B cell proliferation and secretion of IgM and IgA, independent of T cell help (37, 38). Based on our observations, we postulate that the interaction of 4-1BB with 4-1BBL expressed on APCs will enhance the early B-cell response by endowing B cells with a superior ability to proliferate and survive. During the late phase of B cell activation, cognate interaction with T cells will regulate isotype switching, affinity maturation and memory B cell development. While this study establishes the role which 4-1BB plays in protecting activated B cells from activation-induced cell death, the question of whether or not the 4-1BB pathway is involved in these T cell-dependent humoral immune response requires further investigation.

In order to begin to understand the mechanism by which 4-1BB promotes B cell survival, we evaluated expression of intracellular proteins recognized to regulate apoptosis. Our findings indicate that the anti-apoptotic Bcl-2 family proteins Bcl-x1 and Mcl-1 are expressed simultaneously with 4-1BB-mediated protection against activation-induced B cell death. In addition, both Bcl-x1 and Mcl-1 appear to be increased only at the protein level. Protein expression of Mcl-1 is particularly known to be affected post-translationally by either phosphorylation and degradation, or by cleavage through caspases during apoptosis (39, 40). Similarly, cytoprotective cytokines are known to enhance the expression of Mcl-1 through Mcl-1 stabilization (41). Similarly, BAFF also increases the expression of Mcl-1 in human B cells by preventing post-translational changes in this anti-apoptotic molecule (42). Understanding the importance of molecules like Mcl-1 in 4-1BB-mediated B cell survival and Mcl-1 regulation is part of our ongoing studies.

It is to be understood that the invention is not to be limited to the exact configuration as illustrated and described herein. Accordingly, all expedient modifications readily attainable by one of ordinary skill in the art from the disclosure set forth herein, or by routine experimentation therefrom, are deemed to be within the spirit and scope of the invention as defined by the appended claims.

All patents, published patent applications, reference articles, books, journals, manuscripts, manuals, websites and other published materials, as well as drawings, figures, tables as though set forth in full, referred to herein are expressly incorporated herein by reference in their entireties.

### References

1. Schwarz, H., Valbracht, J., Tuckwell, J. et al., ILA, the human 4-1BB homologue, is inducible in lymphoid and other cell lineages, Blood, 85:1043-1052, 1995
2. Schwarz, H., Blanco, F. J., von Kempis, J. et al., ILA, a member of the human nerve growth factor/tumor necrosis factor receptor family, regulates T-lymphocyte proliferation and survival. Blood, 87:2839-2845, 1996.
3. Lin, W., Voskens, C. J., Zhang, X. et al., Fc dependent expression of 4-1BB on human NK cells: insights into “agonistic” effects of anti-4-1BB monoclonal antibodies, Blood, 112(3):699-707, 2008.
4. DeBenedette, M., Shahinian, A, Mak, T. et al., Costimulation of CD28-T lymphocytes by 4-1BB ligand, J. Immunol., 158:551-559, 1997.
5. Pollok, K. E., Kim, Y. J., Hurtado, J. et al., 4-1BB T-cell antigen binds to mature B cells and macrophages, and costimulates anti-mu-primed splenic B cells, Eur. J. Immunol., 24:367-374, 1994.
6. Melero, I., Shuford, W., Newby, S. et al., Monoclonal antibodies against the 4-1BB T-cell activation molecule eradicate established tumors, Nat. Med., 3:682-685, 1997.
7. Ye, Z., Hellstrom, I., Hayden-Ledbetter, M. et al., Gene therapy for cancer using single-chain Fv fragments specific for 4-1BB, Nat. Med., 8:343-348, 2002
8. Chen, S. H., Pham-Nguyen, K. B., Martinet, O. et al., Rejection of disseminated metastases of colon carcinoma by synergism of IL-12 gene therapy and 4-1BB costimulation, Mol. Ther., 2:39-46, 2000.
9. Foell, J., Strahotin, S., O'Neil, S. P. et al., 4-1BB costimulatory T cell receptor engagement reverses acute disease in lupus-prone NZB x NZW Fl mice, J. Clin. Invest., 111:1505-1518, 2003
10. Kim, J., Choi, W. S., La, S. et al., Stimulation with 4-1BB (4-1BB) inhibits chronic graft-versus-host disease by inducing activation-induced cell death of donor CD4+ T cells, Blood, 105:2206-2213, 2005.
11. Seo, S. K., Choi, J. H., Kim, Y. H. et al. 4-1BB-mediated immunotherapy of rheumatoid arthritis, Nat. Med., 10:1088, 2004.
12. Foell, J. L., Diez-Mendiondo, B. I., Diez, O. H. et al., Engagement of the 4-1BB (4-1BB) costimulatory molecule inhibits and reverses the autoimmune process in collagen-induced arthritis and establishes lasting disease resistance, Immunology, 113:89-98, 2004.
13. Lee, J., Lee. E.-N., Kim, E.-Y. et al., Administration of agonistic anti-4-1BB monoclonal antibody leads to the amelioration of inflammatory bowel disease, Immunol. Lett., 101:210-216, 2005.
14. Sun, Y., Chen, H. M., Subudhi, S. K. et al. Costimulatory molecule-targeted antibody therapy of a spontaneous autoimmune disease [comment], Nat. Med., 8:1405-1413, 2002.
15. Zheng, Y., Manzotti, C. N., Liu, M. et al., CD86 and CD80 differentially modulate the suppressive function of human regulatory T cells, J. Immunol., 172:2778-2784, 2004.
16. Kwon, B, S, Hurtadom J. C., Lee, Z. H. et al., Immune Responses in 4-1BB (4-1BB)-Deficient Mice, J. Immunol., 168:5483-5490, 2002
17. Mittler, R., Bailey, T., Klussman, K. et al., Anti-4-1BB monoclonal antibodies abrogate T cell-dependent humoral immune responses in vivo through the induction of helper T cell anergy, J. Exp. Med., 190:1535-1540, 1999.
18. Hong, H., Lee, J., Park, S. et al., A humanized anti-4-1BB monoclonal antibody suppresses antigen-induced humoral immune response in nonhuman primates, J. Immunother., 23:613-621, 2000.
19. Zhu, G., Flies, D. B., Tamada, K. et al., Progressive Depletion of Peripheral B Lymphocytes in 4-1BB (4-1BB) Ligand/I-E{alpha}-Transgenic Mice, J. Immunol., 167:2671-2676, 2001.
20. Futagawa, T., Akiba, H., Kodama, T. et al., Expression and function of 4-1BB and 4-1BB ligand on murine dendritic cells, Int. Immunol., 14:275-286, 2002.
21. Denz, A., Eibel, H, Illges H., Impaired up-regulation of CD86 in B cells of “type A” common variable immunodeficiency patients, Eur. J. Immunol., 30:1069-1077, 2000.
22. Inoue, S., Leitner, W. W., Golding, B., et al., Inhibitory Effects of B Cells on Antitumor Immunity, Cancer Res., 66:7741-7747, 2006
23. Porakishvili, N., Mageed, R., Jamin, C. et al., Recent Progress in the Understanding of B-Cell Functions in Autoimmunity, Scand. J. Immunol., 2001;54:30-38.
24. Toubi, E. and Shoenfeld, Y., The Role of CD40-CD154 Interactions in Autoimmunity and the Benefit of Disrupting this Pathway, Autoimmunity, 7:457-464, 2004.
25. Hirano, T. Cytokines in autoimmune disease and chronic inflammatory proliferative disease, Cytokine Growth Factor Rev., 13:297-8, 2002.
26. Jelinek, D. F. and Lipsky, P. E., The role of B cell proliferation in the generation of immunoglobulin-secreting cells in man, J. Immunol., 130:2597-2604, 1983.
27. Tangye, S. G., Ferguson, A., Avery, D. T. et al. Isotype Switching by Human B Cells Is Division-Associated and Regulated by Cytokines, J. Immunol., 169:4298-4306, 2002.
28. Bernasconi, N. L., Traggiai, E., Lanzavecchia, A. et al., Maintenance of Serological Memory by Polyclonal Activation of Human Memory B Cells, Science, 298:2199-2202, 2002.
29. Fecteau, J. F. and Neron, S., CD40 Stimulation of Human Peripheral B Lymphocytes: Distinct Response from Naive and Memory Cells. J. Immunol., 171:4621-4629, 20031.
30. Lee, H. W., Nam, K. O., Park, S. J., 4-1BB enhances CD8+ T cell expansion by regulating cell cycle progression through changes in expression of cyclins D and E and cyclin-dependent kinase inhibitor p27kipl, Eur. J. Immunol., 33:2133-2141, 2003.
31. Lee, H.-W., Park, S.-J., Choi, B. K. et al., 4-1BB Promotes the Survival of CD8+ T Lymphocytes by Increasing Expression of Bcl-xL and Bfl-1, J. Immunol., 169:4882-4888, 2002.
32. Starck, L., Scholz, C., Dorken, B., Costimulation by 4-1BB/4-1BB inhibits T cell apoptosis and induces Bcl-xL and c-FLIP-1 via phosphatidylinositol 3-kinase and AKT/protein kinase B, Eur. J. Immunol., 35:1257-1266, 2005.
33. Kehry, M. R., Commentary: CD40-mediated signaling in B cells. Balancing cell survival, growth, and death, J. Immunol., 156:2345-2348, 1996.
34. Baumgarth, N., A two-phase model of B-cell activation, Immunol. Rev., 2000; 176:171-180, 2000
35. Snapper, C. M., Mond, J. J., Commentary: A model for induction of T cell-independent humoral immunity in response to polysaccharide antigens, J. Immunol., 157:2229-2233, 1996
36. Dubois, B., Massacrier, C., Vanbervliet, B. et al., Critical Role of IL-12 in Dendritic Cell-Induced Differentiation of Naive B Lymphocytes, J. Immunol., 161:2223-2231, 1998
37. Moore, P. A., Belvedere, O., Orr, A. et al. BLyS: Member of the Tumor Necrosis Factor Family and B Lymphocyte Stimulator, Science, 285:260-263, 1999.
38. Schneider, P., MacKay, F., Steiner, V. et al., BAFF, a Novel Ligand of the Tumor Necrosis Factor Family, Stimulates B Cell Growth, J Exp Med., 189:1747-1756, 1999.
39. Maurer, U., Charvet, C., Wagman, A. S. et al., Glycogen synthase kinase-3 regulates mitochondrial outer membrane permeabilization and apoptosis by destabilization of MCL-1, Mol Cell., 21:749-760, 2006.
40. Weng, C., Li, Y., Xu, D., et al., Specific cleavage of Mcl-1 by caspase-3 in tumor necrosis factor-related apoptosis-inducing ligand (TRAIL)-induced apoptosis in Jurkat leukemia T cells, J. Biol. Chem., 280:10491-10500, 2005.
41. Opferman, J. T., Iwasaki, H., Ong, C. C. et al., Obligate role of anti-apoptotic MCL-1 in the survival of hematopoietic stem cells, Science, 307:1101-1104, 2005.
42. Woodland, R. T., Fox, C. J., Schmidt, M. R. et al., Multiple signaling pathways promote B lymphocyte stimulator dependent B-cell growth and survival, Blood, 111:750-760, 2008.
43. Noelle, R. J., Roy, M., Shepherd, D. M. et al., A 39-kDa protein on activated helper T cells binds CD40 and transduces the signal for cognate activation of B cells, Proc. Nat. Acad. Sci., USA., 89:6550-6554, 1992.
44. Grewal, I. S. and Flavell, R. A., CD40 and CD154 in Cell-Mediated Immunity, Ann. Rev. Immunol., 16:111-135, 1998.
45. Palma, C., Binaschi, M., Bigion, M., 4-1BB and 4-1BB ligand constitutively coexpressed on human T and B leukemia cells signal proliferation and survival, Int. J. Cancer, 108:390-398, 2004.

1-11. (canceled)

12. A method for enhancing immunity of a patient in need thereof, comprising administering to said patient a therapeutically effective amount of a pharmaceutical composition, thereby enhancing immunity of said patient, wherein said patient is suffering from either a B lymphocyte malignancy or a disorder associated with 4-1BB expression; and wherein said pharmaceutical composition comprises an anti-4-1BB antibody and a carrier or a 4-1BB ligand and a carrier.

13. (canceled)

14. The method of claim 12, wherein said disorder associated with 4-1BB expression is a cancer, a B lymphocyte malignancy, an autoimmune disease, asthma, an allergy, or a chronic graft-versus-host disease.

15. The method of claim 14, wherein said autoimmune disease is selected from the group consisting of rheumatoid arthritis (RA), systemic lupus erythematosus (SLE), arteriosclerosis, arthritis, Crohn's disease, Hashimoto's thyroiditis, Addison's disease, juvenile diabetes, diabetes, Grave's disease, myasthenia gravis, Reiter's syndrome, and multiple sclerosis.

16. The method of claim 12, wherein a radioimmunotherapeutic agent or a chemotherapeutic agent is administered prior to, concurrently with, or after administration of the pharmaceutical composition.

17. The method of claim 12, wherein said B lymphocyte malignancy is selected from the group consisting of follicular lymphoma, endemic Burkitt's lymphoma, sporadic Burkitt's lymphoma, non-Burkitt's lymphoma, mucosa-associated lymphoid tissue MALT/MALToma (extranodal), monocytoid B-cell lymphoma (nodal), splenic lymphoma with villous lymphocytes, mantle cell lymphoma, diffuse large cell lymphoma, diffuse mixed large cell lymphoma, immunoblastic lymphoma, primary mediastinal B-cell lymphoma, pulmonary B cell angiocentric lymphoma, and small lymphocytic lymphoma.

18-34. (canceled)

35. A method for determining the responsiveness of a patient suffering from a disease to a therapy, comprising:
(a) determining the amount of 4-1BB or 4-1BB ligand present in a biological sample of a patient after receiving a therapy, and
(b) comparing the amount of 4-1BB or 4-1BB ligand determined in (a) to a control value obtained from a biological sample of said patient prior to receiving the therapy, wherein the result of the comparison of (b) provides a determination of the responsiveness of the patient suffering from the disease to the therapy, and wherein said disease is associated with 4-1BB expression and selected from the group consisting of cancer, B lymphocyte malignancy, autoimmune disease, asthma, allergy and chronic graft-versus-host disease.

36. (canceled)

37. The method of claim 35, wherein said B lymphocyte malignancy is selected from the group consisting of follicular lymphoma, endemic Burkitt's lymphoma, sporadic Burkitt's lymphoma, non-Burkitt's lymphoma, mucosa-associated lymphoid tissue MALT/MALToma (extranodal), monocytoid B-cell lymphoma (nodal), splenic lymphoma with villous lymphocytes, mantle cell lymphoma, diffuse large cell lymphoma, diffuse mixed large cell lymphoma, immunoblastic lymphoma, primary mediastinal B-cell lymphoma, pulmonary B cell angiocentric lymphoma, and small lymphocytic lymphoma.

38. The method of claim 35, wherein said autoimmune disease is selected from the group consisting of rheumatoid arthritis (RA), systemic lupus erythematosus (SLE), arteriosclerosis, arthritis, Crohn's disease, Hashimoto's thyroiditis, Addison's disease, juvenile diabetes, diabetes, Grave's disease, myasthenia gravis, Reiter's syndrome, and multiple sclerosis.

39. A method for evaluating the progression of a disease in a patient, comprising:
(a) determining the amount of 4-1BB or 4-1BB ligand present in a biological sample of a patient, and
(b) comparing the amount of 4-1BB or 4-1BB ligand determined in (a) to a control value obtained from a biological sample of said patient at a date earlier than the date upon which the biological sample of (a) was obtained, wherein the result of the comparison of (b) provides an evaluation of the progression of the disease in the patient, and wherein said disease is associated with 4-1BB expression and selected front the group consisting of cancer, B lymphocyte malignancy. autoimmune disease. asthma, allergy and chronic graft-versus-host disease.

40. (canceled)

41. The method of claim 39, wherein said B lymphocyte malignancy is selected from the group consisting of follicular lymphoma, endemic Burkitt's lymphoma, sporadic Burkitt's lymphoma, non-Burkitt's lymphoma, mucosa-associated lymphoid tissue MALT/MALToma (extranodal), monocytoid B-cell lymphoma (nodal), splenic lymphoma with villous lymphocytes, mantle cell lymphoma, diffuse large cell lymphoma, diffuse mixed large cell lymphoma, immunoblastic lymphoma, primary mediastinal B-cell lymphoma, pulmonary B cell angiocentric lymphoma, and small lymphocytic lymphoma.

42. The method of claim 39, wherein said autoimmune disease is selected from the group consisting of rheumatoid arthritis (RA), systemic lupus erythematosus (SLE), arteriosclerosis, arthritis, Crohn's disease, Hashimoto's thyroiditis, Addison's disease, juvenile diabetes, diabetes, Grave's disease, myasthenia gravis, Reiter's syndrome, and multiple sclerosis.

43-46. (canceled)

47. The method of claim 12, wherein said immunity is innate immunity.

48. The method of claim 12, wherein said immunity is adaptive immunity.
